(19) United States
(12) Patent Application Publication
(10) Pub. No.: US 2004/0214158 A1
(43) Pub. Date: Oct. 28, 2004

(54) Treatment of human papillomavirus (hpv)-infected cells

(76) Inventors: Neerja Sethi, San Francisco, CA (US); Joel Palefsky, Redwood City, CA (US)

Correspondence Address: The Regents of the University of California, 12th Floor, 1111 Franklin Street, Oakland, CA, 94607-5200, US

(21) Appl. No.: 10/276,076

(22) PCT Filed: May 11, 2001
(86) PCT No.: PCT/US01/15407

Related U.S. Application Data
(60) Provisional application No. 60/203,709, filed on May 12, 2000.

(51) Int. Cl. C12Q 1/70; A61K 39/12

(52) U.S. Cl. 435/5; 424/186.1

(57) ABSTRACT

This invention provides a novel gene therapy approach to specifically eliminate keratinocytes, or other cells, expressing early HPV (e.g. HPV 16) genes and which is minimally toxic to HPV-negative cells. In a preferred embodiment this method involves transfecting a mammalian cell with a nucleic acid construct encoding an HPV specific promoter that is induced by an HPV protein where the promoter is operably linked to a nucleic acid comprising a cytotoxic gene such that the cell, when infected with a human papilloma virus induces expression of the cytotoxic gene thereby resulting in the death of said mammalian cell.

### CROSS-REFERENCE TO RELATED APPLICATIONS

This application is a continuation-in-part of U.S. Ser. No. 60/203,709, filed on May 12, 2000, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

In one embodiment, this invention relates to the field of oncology. More particularly this invention pertains to a method of selectively killing HPV-infected epithelial cell.s

### BACKGROUND OF THE INVENTION

Since the late 1970s it has become increasingly clear that infections with human papillomavirus (HPV) are causally implicated in the etiology of anogenital SCC (squamous cell carcinoma) and its precursor, high-grade dysplasia, also known as high-grade intraepithelial neoplasia (IN) or high-grade squamous intraepithelial lesions (SIL). HPV 16 is the most common HPV type found in both cervical and anal SCC (Frisch et al. (1997) New England Journal of Medicine, 337(19): 1350-1358; Walboomers et al. (1999) J. Pathology, 189(1): 12-19). Currently, all therapies for HPV 16-associated lesions rely on ablation or removal of HPV-infected tissue. These methods do not treat HPV infection per se, and virus may be left behind incurring risk of disease recurrence. There is an urgent need for the development of a better treatment strategy for intraepithelial neoplasia that can be implemented to prevent progression to cancer. The goal of this work was to develop a novel gene therapy approach to specifically eliminate keratinocytes expressing early HPV (e.g. HPV-16) genes and which would be minimally toxic to HPV-negative cells.

The regulation of HPV gene expression is complex and is controlled by cellular and viral transcription factors. The expression of HPV oncoproteins E6 and E7 is necessary for immortalization of normal cervical epithelial cells and primary human keratinocytes in vitro and depends on regulatory sequences in the upstream regulatory region (URR) or the long control region (LCR). For most HPV types the LCR is the main transcription regulatory region and contains multiple host transcription-factor binding sites as well as promoter sequences (Bernard and (1994) Arch. Dermatalogy. 130: 210-215; Bouvard et al. (1994) EMBO J. 13: 5451-5459; Chen et al (1997) Cancer Res., 57: 1614-1619; Cripe et al. (1987) EMBO J. 6: 3745; Schwarzet al. (1985) Nature 314: 111; zur Hausen (1991) Virology. 184: 9-13). The origins of replication (ori) sequences are also included in this region.

The HPV E1 and E2 proteins are the viral factors critical for viral replication and transcription (Bernard and (1994) Arch. Dermatalogy. 130: 210-215; Bouvard et al. (1994) EMBO J. 13: 5451-5459; Chen et al. (1997) Cancer Res., 57: 1614-1619; Cripe et al. (1987) EMBO J. 6: 3745; McBride et al. (1991) J. Biol. Chem. 266: 18411-18414). The E2 protein is a transcriptional regulator that binds to a 12-base pair palindromic sequence, ACCN₆GGT (SEQ ID NO:1), which serves as the E2 binding site (E2BS) (Bouvard et al. (1994) EMBO J. 13: 5451-5459; Broker et al. (1989) Cancer Cells. Molecular Diagnostics of Human Cancer by Cold Spring Harbor Laboratory, 197-208; Chen et al. (1997) Cancer Res., 57: 1614-1619; Harris and Botchan (1999) Science 284 (5420): 1673; McBride et al. (1991) J. Biol. Chem. 266: 18411-18414). The LCR from most HPVs includes four E2BS and one E1 binding site. The HPV E2 protein plays a regulatory role in the activation/repression of the LCR promoter and in autoregulating E2 expression. The E2 protein has the ability to either activate or repress HPV promoters, depending on the position of the E2 binding site and on the amount of the full-length E2 protein (Bernard and (1994) Arch. Dermatalogy. 130: 210-215; Broker et al. (1989) Cancer Cells. Molecular Diagnostics of Human Cancer by Cold Spring Harbor Laboratory, 197-208; McBride et al. (1991) J. Biol Chem. 266: 18411-18414). While low levels of full-length E2 protein have been shown to stimulate transcription from the E6 promoter in combination with other host transcriptional factors, high levels of full-length E2 protein may repress the E6 promoter. This feedback mechanism is mostly observed when the HPV genome is maintained in episomal form (Broker et al. (1989) Cancer Cells. Molecular Diagnostics of Human Cancer by Cold Spring Harbor Laboratory, 197-208; Maitland et al. (1998) Journal of pathology, 186(3): 275-80; Stoler et al. (1992) Human Pathology, 23(2): 117-128; zur Hausen (1991) Virology. 184: 9-13).

A study that examined the expression patterns of the HPV 16 E2 transcription factor in low- and high-grade cervical intraepithelial neoplasia (CIN) has shown that E2 expression was highest in CIN I and in koilocytic lesions. E2 expression was detected in superficial layers of the cervical epithelium, as well as in the basal layers in CIN I (Maitland et al. (1998) Journal of Pathology, 186(3): 275-280). Lower expression was observed in CIN II and little in CIN III lesions. In contrast, there was some restoration of E2 expression in invasive carcinomas, although the intracellular distribution was much more diffuse.

### SUMMARY OF THE INVENTION

This invention provides a novel gene therapy approach to specifically eliminate keratinocytes (or other cells) expressing early HPV (e.g., HPV 16). The methods described herein are minimally toxic to HPV-negative cells.

This invention exploits the ability of certain HPV proteins (e.g. E2 protein) to transactivate the viral HPV promoter elements (e.g. HPV-16 promoter elements) to drive expression of an exogenous cytotoxic gene/cDNA (e.g. gene-herpes simplex virus 1 (HSV 1) thymidine kinase (TK)) (see, e.g., Moolten (1994) Cancer Gene Therapy. 1: 279-287; Mullen (1994) Pharmacol. Therapy, 63: 199-207; Nishihara et al. (1998) AntiCancer Res., 1521-1526).

In preferred embodiments, this strategy involves transferring a nucleic acid construct comprising a cytotoxin gene (e.g. the HSV 1-cytotoxin suicide gene) under the control of HPV E2 responsive promoter elements from the LCR into cells of a mammal (human or veterinary) that are infected or that are at risk of infection by HPV. When the cell is infected with HPV, HPV proteins (e.g. E2) are expressed resulting in transactivation of the construct and expression of the cytotoxin either killing the cell or rendering the cell susceptible to one or more drugs (e.g. ganciclovir, acyclovir, etc.).

Thus, in one embodiment, this invention provides a method of selectively inhibiting (growth or proliferation) or killing a cell bearing a human papillomavirus (HPV). The method involves transfecting a mammalian cell with a nucleic acid construct encoding an HPV specific promoter that is induced by an HPV protein where the promoter is operably linked to a nucleic acid comprising a cytotoxic gene whereby the cell, when infected with a human papilloma virus (HPV), induces expression of the cytotoxic gene thereby resulting in the death of the mammalian cell.

In certain embodiments, the transfecting comprises delivery using a vector selected from the group consisting of a retroviral vector, an adeno-associated vector (AAV), an adenoviral vector, a herpes viral vector, and a Sindbis viral vector. The transfecting can comprise comprises using a delivery agent (transfection agent), e.g. an agent selected from the group consisting of a lipid, a liposome, a cationic lipid, and a dendrimer.

The promoter comprising the construct is preferably a promoter up-regulated by an HPV E2 protein. One preferred promoter is from HPV-16. Other preferred promoters include, but are not limited to, promoters from HPVs identified herein in Table 1. Certain preferred promoters comprise a fill-length HPV LCR Certain preferred promoters comprise length of an HPV LCR to induce transcription of a nucleic acid in response to an HPV protein (e.g. an E2 protein). Preferred HPV LCRs include, but are not limited to, LCRs from the HPV identified herein in Table 1. In certain particularly preferred embodiments, the LCR is an HPV-6, HPV-11, or HPV-16 LCR. In certain most preferred embodiments, the promoter is an HPV-6, HPV-11, or HPV-16 LCR promoter.

Preferred cytotoxin genes (cDNAs) include, but are not limited to a ricin gene, an abrin gene, a Pseudomonas exotoxin gene, a diphtheria toxin gene, and a thymidine kinase (tk) gene. In one preferred embodiment the nucleic acid construct comprises an HPV-16 promoter operably linked to a herpes simplex thymidine kinase gene.

Where the cytoxin gene is a tk gene, the method can further involve contacting the cell with ganciclovir (GCV), acyclovir (ACV), or analogues thereof (e.g. valyl esters of GCV and ACV such as valganciclovir, valacyclovir and the like). In certain embodiments, the cell is an epithelial cell, a cancer cell, a cell comprising an intraepithelial neoplasia (IN), a cell comprising an anogenital cancer, a metastatic cell, a cell comprising a solid tumor, a cell comprising a wart, and the like.

In another embodiment, this invention provides a nucleic acid construct comprising an HPV promoter operably linked to a heterologous effector gene. In preferred embodiments, the promoter is a promoter up-regulated by an HPV protein (e.g. an HPV E2 protein). Preferred promoters include, but are not limited to promoters from the HPV idenfied herein in Table 1, more preferably include promoters from HPV-6, HPV-11, and HPV-16. In certain embodiments, the promoter is a full-length HPV LCR. In certain embodiments, the promoter is sufficient length of an HPV LCR to induce transcription of a nucleic acid in response to an E2 protein. In a particularly preferred embodiment, the promoter is an HPV-6, HPV-11, or HPV-16 LCR promoter.

In certain embodiments, the effector is a reporter gene, a cytotoxic gene, a tumor suppressor gene, or an apoptosis gene. Particularly preferred cytotoxic genes include, but are not limited to a ricin gene, an abrin gene, a Pseudomonas exotoxin gene, a diphtheria toxin gene, and a thymidine kinase gene.

In still another embodiments, this invention comprises a mammalian cell comprising one or more of the nucleic acid constructs described herein.

This invention also provides composition comprising a vector comprising the nucleic acid construct as described herein. Preferred vectors include, but are not limited to a retroviral vector, an adeno-associated vector (AAV), an adenoviral vectors, a herpes viral vector, and a Sindbis viral vector. The composition can further comprise a pharmacologically acceptable excipient. In certain embodiments, the composition is a pharmaceutical composition in unit dosage form.

In certain embodiments, this invention provides a composition comprising the nucleic acid construct as described herein in a delivery agent. Preferred delivery agents include, but are not limited to a lipid, a liposome, a cationic lipid, and a dendrimer. The composition can further comprise a pharmacologically acceptable excipient. In certain embodiments, the composition is a pharmaceutical composition in unit dosage form.

This invention also provides methods of treating cells infected with HPV. The methods involve transfecting the cells with the nucleic acid construct(s) described herein. The construct is preferably transfected in sufficient concentration to produce a lethal concentration of a cytotoxin in the cells. The method can further involve contacting said cells with with ganciclovir (GCV) or acyclovir (ACV) or derivatives or analogues thereof.

Also provided are kits for selectively killing cells infected with HPV. The kits preferably comprise a container containing one or more of the constructs and/or compositions described herein.

This invention also provides methods of selectively labeling a cell bearing a human papillomavirus (HPV). These methods involve transfecting a mammalian cell with a nucleic acid construct encoding an HPV specific promoter that is induced by an HPV protein wherein the promoter is operably linked to a nucleic acid comprising a reporter gene whereby the cell, when infected with a human papilloma virus (HPV) induces expression of the reporter gene thereby labeling the mammalian cell. The transfecting can comprise delivery using a vector (e.g. a retroviral vector, an adeno-associated vector (AAV), an adenoviral vectors, a herpes viral vector, a Sindbis viral vector, etc.). The transfecting can comprise using a delivery agent (e.g. a lipid, a liposome, a cationic lipid, and a dendrimer).

In preferred embodiments, the promoter is a promoter up-regulated by an HPV E2 protein. Preferred promoters include, but are not limited to, promoters from an HPV listed herein in Table 1, more preferably a promoter from HPV-6, HPV-11 or HPV-16. Certain preferred promoters include a full-length HPV LCR. Certain preferred promoters include a sufficient length of an HPV LCR to induce transcription of a nucleic acid in response to an E2 protein. Particulary preferred promoters include, but are not limited to, an HPV-6, HPV-11 or HPV-16 LCR. Preferred reporter genes include, but are not limited to an enzymatic reporter, a colorimetric reporter, a luminescent reporter, and a fluorescent reporter. Particularly preferred reporters include an Fflux gene or a green fluorescent protein gene. In certain embodiments, the cell is an epithelial cell, a cancer cell, a cell comprising an intraepithelial neoplasia (IN), a cell comprising an anogenital cancer, a metastatic cell, a cell comprising a solid tumor, a cell comprising a wart, and the like.

Definitions

“Transfection” is used herein to mean the delivery of a nucleic acid to a target cell, such that the nucleic acid enters the cell. It will be understood that the term “nucleic acid” includes both DNA and RNA without regard to molecular weight, and the term “expression” means any manifestation of the functional presence of the nucleic acid within the cell, including without limitation, transcription and/or translation and both transient expression and stable expression.

“Delivery” is used to denote a process by which a desired compound is transferred to a target cell such that the desired compound is ultimately located inside the target cell or in, or on the target cell membrane. In many uses of the compounds of the invention, the desired compound is not readily taken up by the target cell and delivery via lipid aggregates is a means for getting the desired compound into the cell. In certain uses, especially under in vivo conditions, delivery to a specific target cell type is preferable and can be facilitated by compounds of the invention.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “heterologous” as it relates to nucleic acid sequences such as coding sequences and control sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

The term “operably linked” as used herein refers to linkage of a promoter to a nucleic acid sequence such that the promoter mediates/controls transcription of the nucleic acid sequence.

The term “induce” expression refers to an increase in the transcription and/or translation of a gene or cDNA.

### BRIEF DESCRIPTION OF THE DRAWINGS

FIG. 1 illustrates one embodiment of the methods described herein. Cell lines expressing HPV 16 E2 protein were transfected with a plasmid expressing HSV 1-TK under the control of HPV 16 LCR. Treatment of these cells with the prodrugs GCV or ACV causes cell death due to the formation of toxic phosphorylated GCV (GCP) or phosphorylated ACV (ACP).

FIG. 2 illustrates a gene therapy vector that can replicate and be maintained extrachromosomally

FIG. 3A shows a schematic representation of the HPV 16 viral LCR upstream of the early promoter P97. E2 boxes represent the binding sites for the E2 protein. The LCR includes binding sites for a number of host transcription factors including AP1 and NF1 which are shown. The enhancer box includes the binding sites for transcription factors in the region of the major constitutive enhancer of HPV 16. The numbers represent the nucleotide positions in the HPV16 genome. FIG. 3B shows a schematic representations of the plasmids used in this study. Plasmids pNSXH-4, pNSGLTK-8, and pNSGL0 are all derived from pGL3 Basic Vector (Promega) which carries a luciferase-luc+gene.

FIG. 4 shows RT-PCR analysis of HSV 1-TK and HPV-16 E2 in CaSki cells seven days after transfection with pNSGLTK-8. Lanes A, B, and C show RT-PCR products amplified using HSV 1-TK specific primers which give a product of approximately 288 base pairs. Lane (A) mRNA from HSC3 cells, (B) mRNA from CaSki cells, and (C) mRNA from CaSki cells with no RT as a negative control. Lanes D, E, and F show RT-PCR products amplified using HPV 16 E2-specific primers which give a product of approximately 216 base pairs. Lane (D) mRNA from HSC3 cells, (E) mRNA from CaSki cells, and (E) mRNA from CaSki cells with no RT as a negative control.

FIG. 5 illustrates the stimulation of luciferase expression by HPV 16-positive and HPV 16-negative cell lines. CaSki, HeLa and HSC3 cell lines were transfected with pNSXH-4 plasmid carrying the luciferase gene under the control of the HPV 16 LCR promoter. The activity from the parental plasmid pGL3 Basic (Promega) was used as a reference. 24 hours after transfection, luciferase activity was determined and plotted on a log scale. Values represent the average of triplicate determinations; bars represent standard deviation.

FIG. 6 shows dose dependent in vitro cytotoxicity of ganciclovir (GCV) on CaSki cells. The cells were transfected with pNSGLTK-8 or pNSGL0, or mock-transfected with no DNA before being treated with 0, 5, 10, and 20 μg/ml concentrations of GCV. Cell viability was measured by the MTS cell proliferation assay after six days of treatment. Values represent the average of triplicate determinations; bars represent standard deviation.

FIG. 7 shows a time-course analysis of in vitro cytotoxicity of 20.0 μg/ml of GCV on CaSki cells. The cells were transfected with pNSGLTK-8 or pNSGL0, or mock-transfected with no DNA before being treated with GCV. Cell viability was measured by the MTS cell proliferation assay at baseline and after two, four, six and ten days of GCV treatment. Values represent the average of triplicate determinations; bars represent standard deviation.

FIG. 8 shows dose dependent in vitro cytotoxicity of acyclovir (ACV) on CaSki cells. The cells were transfected with pNSGLTK-8 or pNSGL0, or mock-transfected with no DNA before being treated with 0, 10, 20, and 30 μg/ml concentrations of ACV. Cell viability was measured by the MTS cell proliferation assay after ten days of treatment. Values represent the average of triplicate determinations; bars represent standard deviation.

FIG. 9 shows in vitro cytotoxicity of 20.0 μg/ml of GCV on three different cell lines: CaSki, SiHa and HSC3. The cells were transfected with pNSGLTK-8 or mock-transfected with no DNA before being treated with GCV. Cell viability was measured by the MTS cell proliferation assay after six days of treatment with GCV. Values represent the average of triplicate determinations; bars represent standard deviation.

FIGS. 10A-10D show the induction of apoptosis in CaSki cells transfected with pNSGLTK-8 expressing the HSV 1-1-TK gene under the control of the HPV 16 LCR promoter and exposed to 20.0 μg/ml GCV for six days. The cells were analyzed using a modified biotinylated TUNEL stain, which stains the fragmented DNA dark brown. The cells were analyzed at following times after exposure to GCV: FIG. 10A: day zero, showing basal level apoptosis; FIG. 10B: two days, presence of a large number of cells with dark brown staining fragmented DNA, FIG. 10C: four days, cells with highly condensed nuclei or very dark brown fragmented DNA, and FIG. 10D: six days, very few cells are seen because of cell death. Cells showing apoptotic morphology of punched out cytoplasm and apoptotic nuclei.

FIGS. 11A-11C illustrate induction of apoptosis in SiHa cells transfected with pNSGLTK-8 expressing the HSV 1-TK gene under the control of the HPV 16 LCR promoter and exposed to 20.0 μg/ml GCV for ten days. The cells were analyzed using a modified biotinylated TUNEL stain which stains the fragmented DNA dark brown. The cells were analyzed at following times after exposure to GCV: FIG. 11A: day zero, showing basal level apoptosis; FIG. 11B: eight days, cells showing dark brown apoptotic nuclei, and FIG. 11C: ten days, cells showing apoptotic nuclei, cells making fine intercellular connections and few cells containing apoptotic bodies can be seen.

FIGS. 12A-12D show induction of apoptosis in CaSki cells transfected with pNSGLTK-8 expressing the HSV 1-TK gene under the control of the HPV16 LCR promoter and exposed to 20.0 μg/ml ACV for ten days. The cells were analyzed using a modified biotinylated TUNEL stain, which stains the fragmented DNA dark brown. The cells were analyzed at following times after exposure to ACV: FIG. 12A: day zero, showing basal level apoptosis, FIG. 12B: two days, presence of a large number of cells with dark brown staining fragmented DNA, FIG. 12C: six days, cells showing apoptotic morphology of punched out cytoplasm and apoptotic nuclei, and FIG. 12D: ten days, showing a decrease in number of cells due to cell death. The remaining cells show apoptotic nuclei and some apoptotic bodies can be seen.

FIG. 13 shows a sequence alignment of four papillomavirus E2 proteins, HPV 16, HPV 18, HPV 11 and bovine papillomavirus (HPV)-1 showing the conservation of the transactivation domain residues.

FIG. 14 shows the survival of HPV-positive and HPV-negative cells transfected with an HSV1-TK construct and exposed to varying levels of ganciclovir. The HPV-positive cells were CaSki cells. The HPV negative cells were HSC3—a human oral cancer cell line, MDCK—Madin-Darby canine kidney cell line, VERO— African Green Monkey kidney cell line, and Human oral squamous cell carcinoma cell, SSC9.

FIGS. 15A, 15B and 15C illustrate the detection of the HSV1-TK construct (pNSGLTK-8) in transfected cells. FIG. 15A shows nuclear staining of cells by PI. FIG. 15B shows anti-HSV1-TK staining. FIG. 15C, the merge, shows nuclear and cytoplasmic distribution of HSV1-TK protein.

FIGS. 16A, 16B, and 16C illustrate the detection of the HSV1-TK construct (pNSGLTK-8) in apoptotic cells treated with ganciclovir. FIG. 16A shows nuclear staining of cells by PI. FIG. 16B shows anti-HSV1-TK staining. FIG. 16C, the merge, shows nuclear and cytoplasmic distribution of HSV1-TK protein.

### DETAILED DESCRIPTION

This invention provides a novel gene therapy approach to specifically eliminate keratinocytes, or other cells (e.g., high-grade dysplasia, also known as high-grade intraepithelial neoplasia (IN) or high-grade squamous intraepithelial lesions (SIL)), expressing early HPV (e.g. HPV 16) genes. The methods of this invention provide rapid detection and/or effective killing of cells infected with HPV (e.g. cells expressing HPV proteins) and are minimally toxic to HPV-negative cells.

The methods of this invention exploit the ability of HPV proteins (eg. E2 protein) to transactivate a viral promoter (e.g. the viral HPV 16 promoter elements) to drive expression of an exogenous cytotoxin and/or an exogenous reporter gene (e.g., a gene-herpes simplex virus 1 (HSV 1)-thymidine kinase (TK) (32, 33, 34)). In a preferred embodiment, the methods involve transfer of a cycotoxin and/or reporter gene into cells that are or that can be infected with HPV.

If the transfected cell is infected with HPV or becomes infected with HPV, the expressed HPV proteins induce expression of the gene(s) under control of the viral promoter. The gene(s) typically express a cytotoxin that either kills the subject cell or that renders the cell susceptible to a cytotoxic agent (e.g., ganciclovir, acyclovir, etc.). Alternatively, or in addition, the heterologous gene(s) can encode one or more detectable labels allowing ready identification of the infected cells and/or confirmation of the presence of HPV in the subject cells.

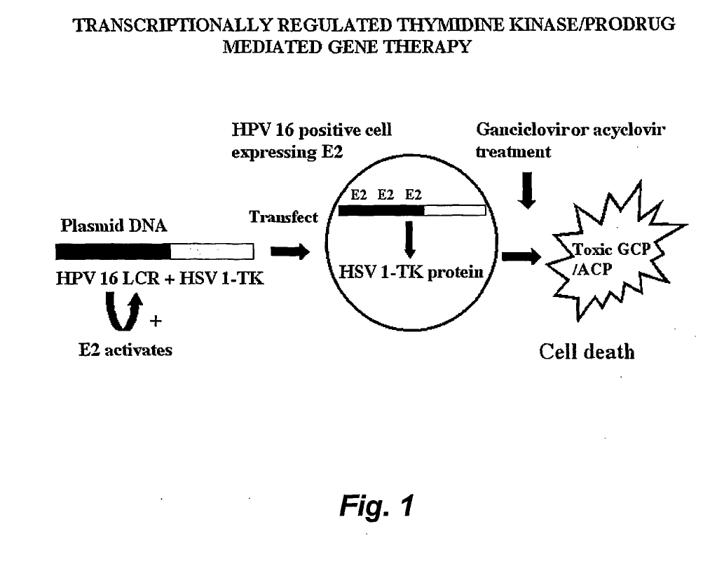

In certain preferred embodiments, the heterologous gene is the cytotoxic thymidine kinase gene (e.g. the HSV 1-TK gene). The gene is placed under the control of the HPV E2 E2-responsive promoter elements from the HPV LCR. Cells expressing E2 express the HSV 1-TK gene rendering them sensitive to nontoxic prodrugs such as ganciclovir (GCV) or acyclovir (ACV). The HSV 1-TK protein in transfected cells phosphorylates GCV or ACV into mono-phosphorylated GCV or ACV, respectively, which is then triphosphorylated by cellular kinases. Triphosphorylated GCV or ACV induces cell death upon incorporation into cellular DNA. This scheme is depicted as a model in FIG. 1.

Selective upregulation of the cytotoxin represents a new, HPV-specific approach to the treatment of squamous cell carcinoma (SCC) precursors. We believe that the treatment of anogenital epithelium might be an ideal indication for the success of this approach given the relatively accessible mucosal location of the lesions and the relative ease of drug delivery to this site.

It is noted however, that the methods of this invention are not limited to HPV-16. Rather, the methods are applicable to essentially any HPV infection and associated pathology. Some representative HPV types and associated clinical diseases are illustrated in Table 1.

TABLE 1
Representative HPV type and associated clinical disease.

| Clinical Diseases | HPV Types |
|---|---|
| Verruca vulgaris (commonwarts) | 2, 4, 29, 57 |
| Verruca plantaris and plana (deep plantar and | 1, 2, 4, 10 |
| palmar warts) | |
| Epidermodysplasia verruciformis | 19-25, 36, 46, 47, 50 |
| Anogenital condyloma acuminatum | 6, 11, 42, 54 |
| Cervical intraepithelial neoplasia and/or cervical | 16, 18, 30, 31, 33, |
| carcinoma | 34, 35, 39, 40, 42, |
| | 43, 45, 51, 52, 56, 58 |
| Oral lesions, laryngeal carcinoma | 30, 40 |
| Cutaneous wart in renal transplant recipients | 27 |

A number of promoters are suitable for practice of the methods of this invention. In preferred embodiments, any promoter that is responsive to the presence of an HPV protein is suitable for use in the constructs of this invention. Such promoters are readily derived from wild-type or modified HPV.

The regulation of HPV gene expression is is controlled by cellular and viral transcription factors. The expression of HPV oncoproteins E6 and E7, for example, is necessary for immortalization of normal cervical epithelial cells and primary human keratinocytes in vitro and depends on regulatory sequences in the upstream long control region (LCR).

For most HPV types the upstream regulatory region (URR) or LCR is the main transcription regulatory region and contains many host transcription-factor binding sites as well as the promoter sequences (Bernard and Apt (1994) Dermatalogy, 130: 210-215; Bouvard et al. (1994) EMBO J., 13: 5451-5459; Chen et al. (1997) Cancer Res. 57:1614-1619; Cripe et al. (1987) EMBO J., 6: 3745; Romanczuk et al. (1990) J. Virol., 70: 1602-1611; Schwarz et al. (1985) Nature 314: 111; zur Hausen (1991) Virology, 184: 9-13). The origins of replication (ori) sequences are also included in this region. The HPV E1 and E2 proteins are the viral factors critical for viral replication and transcription (Bernard and Apt (1994) Dermatalogy, 130: 210-215; Bouvard et al. (1994) EMBO J., 13: 5451-5459; Broker et al. (1989) Cancer Cells. Molecular Diagnostics of Human Cancer by Cold Spring Harbor Laboratory, 197-20; Cripe et al. (1987) EMBO J., 6: 3745; Dowhanick et al. (1995) J. Virol, 69(12): 7791-7799; Steger et al. (1996) Meth. Enzymology, 274: 173-185; Thierry and Yaniv (1987) EMBO J., 6: 6655-6666). The E1 protein and the E2 protein are required for the origin recognition and for replication initiation. The E2 protein is also the critical transcription regulator that binds to a 12 base pair palindromic sequence, ACCN6GGT (SEQ ID NO:1), which serves as the E2 binding site (E2BS) (Bouvard et al. (1994) EMBO J., 13: 5451-5459; Broker et al. (1989) Cancer Cells. Molecular Diagnostics of Human Cancer by Cold Spring Harbor Laboratory, 197-208; Chen et al. (1997) Cancer Res. 57:1614-1619; Harris and Botchan (1999) Science 284(5420): 1673; McBride et al. (1991) J. Biol. Chem. 266: 18411-18414). The LCR from most HPVs includes four E2 binding sites and one E1 binding site.

HPV E2 protein has the ability to either activate or repress HPV promoters, depending on the position of the E2 binding site or the amount of the full-length E2 protein (Bernard and Apt (1994) Dermatalogy, 130: 210-215; Broker et al. (1989) Cancer Cells. Molecular Diagnostics of Human Cancer by Cold Spring Harbor Laboratory, 197-208; Dowhanick et al. (1995) J. Virol., 69(12): 7791-7799; McBride et al. (1991) J. Biol. Chem. 266: 18411-18414; Romanczuk et al. (1990) J. Virol., 70: 1602-1611; Thierry and Yaniv (1987) EMBO J. 6: 6655-6666). Low levels of full-length E2 protein have been shown to stimulate transcription from the E6 promoter in combination with other host transcriptional factors.

The E2 protein plays a critical balancing role in fine-tuning the activation/repression of the E6 promoter, in autoregulating E2 levels and also in controlling the levels of the E6 and E7 oncoproteins. This feedback mechanism is mostly observed in basal or parabasal cells during early stages of infection when the HPV genome is maintained in episomal form (Broker et al. (1989) Cancer Cells. Molecular Diagnostics of Human Cancer by Cold Spring Harbor Laboratory, 197-208; Maitland et al. (1998) J. Pathol., 186(3):275-280; Stoler et al. (1992) Human Pathology. 23(2): 117-128; zur Hausen (1991) Virology, 184: 9-13). A study that examined the expression patterns of the HPV 16 transcription factor E2 in low- and high-grade CIN has shown that E2 expression was highest in CIN I and in koilocytic lesions. Lower expression was observed in CIN II and little in CIN III lesions. In contrast, there was some restoration of E2 expression in invasive carcinomas, although the intracellular distribution was much more diffuse. E2 expression was detected in superficial layers of the cervical epithelium, as well as in the basal layers in CIN I (Maitland et al. (1998) J. Pathol., 186(3):275-280).

In preferred embodiments, this invention utilizes promoters responsive to HPV proteins, more preferably responsive to the HPV E1 or E2 proteins. Most preferably the promoter is a promoter responsive (e.g. induced and/or upregulated by) E2. In particularly preferred embodiments, the promoter comprises a full-length HPV LCR In certain embodiments, the promoter comprises sufficient length of an HPV LCR to induce transcription of a nucleic acid in response to an E1 and or E2 protein, most preferably in response to E2 protein. In particularly preferred embodiments. the promoter comprises a full-length HPV-16 LCR or a fragment thereof.

The invention, however, is not limited to the use of an HPV 16 promoter. Similar promoters from other human papillomavirus strains (e.g. HPV 6, HPV 11, etc.) are also suitable for the methods of this invention This method may therefore be used to treat condyloma acuminatum (warts) associated with HPV 6 or 11. Lesions ranging from condyloma to dysplasia and cancer may be treated using the invention, targeting the range of HPV types associated with these lesions.

In certain embodiments, this invention utilizes HPV, gene therapy vectors that can replicate and be maintained extrachromosomally. Low level expression of E1 and E2 from the LCR ensure low copy number and the absence of transforming effects on the host cells. Addition of multiple E2 binding sites ensures that the vector is maintained as an episome when either viral origin (included in the LCR) or a cellular origin is used.

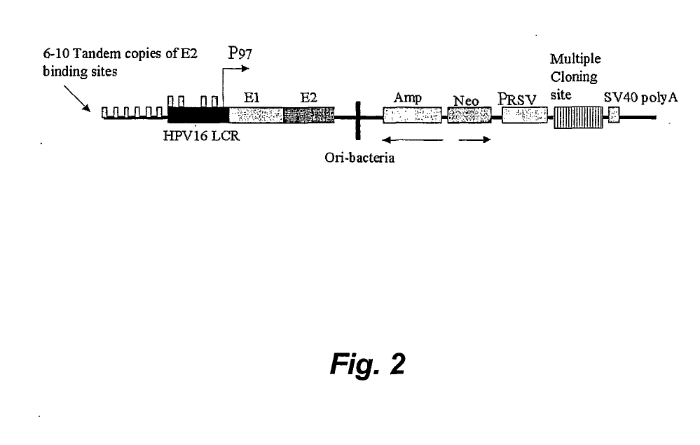

Such vectors are not be affected by the constraints of the levels of the E2 protein and can also be maintained long-term. Such vectors are based on the idea that the viral genome interacts with the host chromatin via the E2 protein (the transactivation-E2TA domain of E2) and E2 binding sites and this interaction ensures equal and stable segregation of the plasmid DNA to daughter cells. One such vector is illustrated in FIG. 2. This construct is similar to those based on EBNA1 from EBV using the oriP which has multiple EBNA1 binding sites. EBNA1 is very similar in function to E2-TA but with no sequence similarity. Both viruses can replicate and exist as extrachromosomal forms. Both proteins have similar DNA binding motifs and both viruses can associate with condensed chromosomes during mitosis and possibly result in longer retention in the cells even in the absence of replication.

A wide variety of cytotoxin and/or reporter genes (cDNAs) can be used in the methods of this invention. Suitable toxins in this regard include, but are not limited to, holotoxins, such as abrin, ricin, modeccin, Pseudomonas exotoxin A, Diphtheria toxin, pertussis toxin and Shiga toxin; and A chain or “A chain-like” molecules, such as ricin A chain, abrin A chain, modeccin A chain, the enzymatic portion of Pseudomonas exotoxin A, Diphtheria toxin A chain, the enzymatic portion of pertussis toxin, the enzymatic portion of Shiga toxin, gelonin, pokeweed antiviral protein, saporin, tritin, barley toxin, cnidarian and snake venom peptides, and the like. Ribosomal inactivating proteins (RIPs), naturally occurring protein synthesis inhibitors that lack translocating and cell-binding ability, are also suitable for use herein.

Another well known cytotoxin is thymidine kinase (e.g., herpes simplex thymidine kinase “HSV-tk”) gene, the product of which is cytotoxic to cells when cells are grown in the presence of ganciclovir or acyclovir. Typically the thymidine kinase, or a gene encoding thymidine kinase is delivered to the target cell. Administration of the drug ganciclovir, acyclovir, or analogues thereof, and the like, will cause the selective killing harboring the tk protein.

In certain particularly preferred embodiments ganciclovir or acyclovir are administered orally. Preferred forms of these drugs for oral delivery include, but are not limited to, valyl esters of GCV and ACV (e.g. valganciclovir and valacyclovir, etc.) which have better bioavailibilty in the systemic form. It is noted that, in clinical trials, valganciclovir, the valyl ester of ganciclovir, has been shown to enhance the bioavailability of ganciclovir when taken orally by patients (Sugawara et al. (2000) J. Pharm. Sci., Jun, 89(6): 781-789.)

The above-described cytotoxins are simply illustrative. Numerous other cytotoxins are well known to those of skill in the art.

In addition to, or instead of cytotoxins, the methods of this invention can be used to specifically express one or more reporter genes in cells infected with HPV and thereby identify and/or localize infected cells. As used herein, a reporter gene refers to gene or cDNA that expresses a product that is detectable by spectroscopic, photochemical, biochemical, immunochemical, electrical, optical or chemical means. Useful labels in this regard include, but are not limited to fluorescent proteins (e.g. green fluorescent protein (GFP), red fluorescent protein (RFP), etc.) enzymes (e.g., horse radish peroxidase, alkaline phosphatase β-galactosidase, and others commonly used in an ELISA), and the like.

In addition, it will be recognized that the cytotoxin gene or the reporter gene need not be so limited and virtually any “effector” gene can be used. Such effectors include, but are not limited to, apoptosis-inducing gene (e.g. P53, P73, Bax, Bad, FADD, a caspase (e.g. Casp3, Casp9, Apaf1, etc.), etc.), tumor suppressors, and the like.

In practice, the methods of this invention involve transfecting a mammalian cell (e.g. one or more cells harboring an HPV or at risk for HPV infection) with a nucleic acid construct encoding an HPV specific promoter that is induced by an HPV protein. The HPV promoter is operably linked to a nucleic acid encoding a cytotoxin and/or a reporter. If the cell harbors HPV or is infected with HPV, the HPV protein induces expression of the nucleic acid under control of the HPV-specific promoter resulting in expression of the cytotoxin and/or label.

The methods are well suited to the treatment of cancerous or pre-cancerous cells. In particular the methods are applied to keratinocytes, more particularly to cells of anogenital SCC (squamous cell carcinoma) and its precursor, high-grade dysplasia, also known as high-grade intraepithelial neoplasia (IN) or high-grade squamous intraepithelial lesions (SIL). Warts may also be treated using the invention.

It is noted that the treatment of anogenital epithelium is ideal for application of the methods of this invention given the relatively accessible mucosal location of the lesions and the relative ease of drug delivery to this site. Moreover, it is further note that topical transfection of cells with heterologous nucleic acids (e.g. antisense molecules) is routinely accomplished (see, e.g., Wraight et al. (2000) nature Biotechnology, 18: 521-525). In addition, transfection reagents suitable for transfection cells by topical application are also well known to those of skill in the art and many are commercially available (e.g. cationic lipids, lipofectamine™, Chariot™, etc.).

The methods of this invention need not be limited to the treatment of cancerous or pre-cancerous cells. The methods are well suited in the treatment of any condition in which HPV infection is a component of the etiology. Thus, for example in certain embodiments, the methods of this invention can be used to target HPV 6, and/or HPV 11 the human papillomaviruses associated with genital warts. The cytotoxin nucleic acid construct can be embodied in a cream or ointment (e.g. containing a transfection agent such as a liposome cationic lipid, starburst dendrimer, etc.). The subject applies the cream or ointment to the warts thereby transfecting the construct into the infected cells. Subsequent administration of ganciclovir (GCV) or acyclovir (ACV) or analogues thereof (e.g. valganciclovir, valacyclovir, etc.) results in killing of the infected (wart) cells.

The constructs of this invention can be delivered according to any of a wide number of methods well known to those of skill in the art. In a preferred embodiment, the nucleic acid(s) encoding the reporter and/or cytotoxic gene under control of the HPV promoter are provided as (e.g. cloned into) gene therapy vectors that are competent to transfect cells (such as human or other mammalian cells) in vitro and/or in vivo.

Many approaches for introducing nucleic acids into cells in vivo, ex vivo and in vitro are known to those of skill in the art. These include, but are not limited to lipid or liposome based gene delivery (see, e.g., WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414), electroporation, calcium phosphate transfection, viral vectors, biolistics, microinjection, dendrimer conjugation, and the like. In particularly preferred embodiments, transfection is by means of replication-defective retroviral vectors (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson (1992) Science 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 3144; Haddada et al. (1995) in Current Topics in Microbiology and Immunology, Doerfler and Böhm (eds) Springer-Verlag, Heidelberg Germany, and Yu et al., (1994) Gene Therapy, 1:13-26.

Widely used vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g. Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include herpes virus, lentivirus, and vaccinia virus.

In one particularly preferred embodiment, retroviruses (e.g. lentiviruses) are used to transfect the target cell(s) with nucleic acids comprising a reporter and/or cytotoxic gene under control of the HPV promoter. Retroviruses, in particular lentiviruses (e.g. Hv, SIV, etc.) are particularly well suited for this application because they are capable of infecting a non-dividing cell. Methods of using retroviruses for nucleic acid transfection are known to those of skill in the art (see, e.g., U.S. Pat. No. 6,013,576).

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. In preferred embodiments, a helper virus need not be utilized for the production of the recombinant retrovirus since the sequences for encapsidation can be provided by co-transfection with appropriate vectors.

The retroviral genome and the proviral DNA have three genes: the gag, the pol, and the env, which are flanked by two long terminal repeat (LIR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vit, vpr, tat, rev, vpu, nef; and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

In one preferred embodiment, the invention provides a recombinant retrovirus capable of infecting a non-dividing cell. The recombinant retrovirus comprises a viral GAG, a viral POL, a viral ENV, a heterologous nucleic acid sequence operably linked to a regulatory nucleic acid sequence, and cis-acting nucleic acid sequences necessary for packaging, reverse transcription and integration, as described above. It should be understood that the recombinant retrovirus of the invention is capable of infecting dividing cells as well as non-dividing cells.

In preferred embodiments, the recombinant retrovirus is therefore genetically modified in such a way that some of the structural, infectious genes of the native virus (e.g. env, gag, pol) have been removed and replaced instead with a nucleic acid sequence to be delivered to a target non-dividing cell (e.g., a sequence encoding the reporter and/or cytotoxic gene under control of the HPV promoter). After infection of a cell by the virus, the virus injects its nucleic acid into the cell and the retrovirus genetic material can, optionally, integrate into the host cell genome. Methods of making and using lentiviral vectors are discussed in detail in U.S. Pat. Nos. 6,013,516, 5,932,467, and the like.

In another preferred embodiment, the reporter and/or cytotoxic gene under control of the HPV promoter are placed in an adenoviral vector suitable for gene therapy. The use of adenoviral vectors is described in detail in WO 96/25507. Particularly preferred adenoviral vectors are described by Wills et al. (1994) Hum. Gene Therap. 5: 1079-1088. Typically, adenoviral vectors contain a deletion in the adenovirus early region 3 and/or early region 4 and this deletion may include a deletion of some, or all, of the protein IX gene. In one embodiment, the adenoviral vectors include deletions of the E1a and/or E1b sequences.

A number of different adenoviral vectors have been optimized for gene transfer. One such adenoviral vector is described in U.S. Pat. No. 6,020,191. This vector comprises a CMV promoter to which a transgene may be operably linked and further contains an E1 deletion and a partial deletion of 1.6 kb from the E3 region. This is a replication defective vector containing a deletion in the E1 region into which a transgene (e.g. the β subunit gene) and its expression control sequences can be inserted, preferably the CMV promoter contained in this vector. It further contains the wild-type adenovirus E2 and E4 regions. The vector contains a deletion in the E3 region which encompasses 1549 nucleotides from adenovirus nucleotides 29292 to 30840 (Roberts et al. (1986) Adenovirus DNA, in Developments in Molecular Virology, W. Doerfler, ed., 8: 1-51). These modifications to the E3 region in the vector result in the following: (a) all the downstream splice acceptor sites in the E3 region are deleted and only mRNA a would be synthesized from the E3 promoter (Tollefson et al. (1996) J, Virol. 70:2 296-2306, 1996; Tollefson et al. (1996) Virology 220: 152-162,); (b) the E3A poly A site has been deleted, but the E3B poly A site has been retained; (c) the E3 gp19K (MHC I binding protein) gene has been retained; and (d) the E3 11.6K (Ad death protein) gene has been deleted.

Such adenoviral vectors can utilize adenovirus genomic sequences from any adenovirus serotypes, including but not limited to, adenovirus serotypes 2, 5, and all other preferably non-oncogenic serotypes.

Alone, or in combination with viral vectors, a number of non-viral vectors are also useful for transfecting cells with reporter and/or cytotoxic genes under control of the HPV promoter. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. Liposomes entrap DNA by one of two mechanisms which has resulted in their classification as either cationic liposomes or pH-sensitive liposomes. Cationic liposomes are positively charged liposomes that interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2,3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycations suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å, and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

Vectors (e.g., retroviruses, adenoviruses, liposomes, etc.) containing therapeutic nucleic acids can be administered directly to the organism for transduction of cells in vivo. Administration is by any of the routes normally used for introducing a molecule into ultimate contact with blood or tissue cells. The nucleic acids are administered in any suitable manner, preferably with pharmaceutically acceptable carriers. Suitable methods of administering such packaged nucleic acids are available and well known to those of skill in the art.

Pharmaceutically acceptable carriers are determined in part by the particular composition being administered, as well as by the particular method used to administer the composition. Accordingly, there is a wide variety of suitable formulations of pharmaceutical compositions of the present invention.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

The packaged nucleic acids, alone or in combination with other suitable components, can be made into aerosol formulations (i.e., they can be nebulized) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of packaged nucleic acid can be presented in unit-dose or multi-dose sealed containers, such as ampules and vials.

Certain preferred embodiments, contemplate topical administration. Such embodiments, include formulations such as creams, gels, foams, poultices, dermal or transdermal patches, and the like.

The pharmaceutical formulations can be prepared from sterile powders, granules, and tablets of the kind previously described. Cells transduced by the packaged nucleic acid as described above in the context of ex vivo therapy can also be administered intravenously or parenterally as described above.

The dose administered to a patient, in the context of the present invention, should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment, the physician evaluates circulating plasma levels of the vector, vector toxicities, progression of the disease, and the production of anti-vector antibodies. The typical dose for a nucleic acid is highly dependent on route of administration and gene delivery system. Depending on delivery method the dosage can easily range from about 1 μg to 100 mg or more. In general, the dose equivalent of a naked nucleic acid from a vector is from about 1 μg to 100 μg for a typical 70 kilogram patient, and doses of vectors which include a viral particle are calculated to yield an equivalent amount of therapeutic nucleic acid.

Dosages of a viral vector of the invention which can be used in providing a transgene contained in a vector to an individual for persistent expression of a biologically active protein encoded by the transgene and to achieve a specific phenotypic result range from approximately 10⁸ infectious units (I.U.) to 10¹¹ I.U. for humans.

For administration, transduced cells of the present invention can be administered at a rate determined by the LD₅₀ of the vector, or transduced cell type, and the side-effects of the vector or cell type at various concentrations, as applied to the mass and overall health of the patient. Administration can be accomplished via single or divided doses as described below.

It is especially advantageous to formulate parenteral compositions in dosage unit form for case of administration and uniformity of dosage. Dosage unit form as used herein refers to physically discrete units suited as unitary dosages for the subjects to be treated, each unit containing a predetermined quantity of active ingredient calculated to produce the specific phenotypic result in association with the required physiological carrier. The specification for the novel dosage unit forms of the invention are dictated by and directly depend on the unique characteristics of the vector used in the formulation and the limitations inherent in the art of compounding. The principal active ingredient (e.g., the viral vector) is compounded for convenient and effective administration with the physiologically acceptable carrier in dosage unit form as discussed above.

Maximum benefit and achievement of a specific phenotypic result from the administration of a vector of this e invention may require repeated administration. Such repeated administration may involve use of the same vector, or, alternatively, may involve the use of different vectors in order to alter viral antigen presentation and decrease host immune response.

Where the methods of this invention entail administration of a drug in addition to the construct described above (e.g. ganciclovir, acyclovir, etc.) the drug is administered in accordance with standard practices known to those of skill in the art. Thus, for example dosages for acyclovir, ganciclovir, and the like are well known to those of skill in the art. In addition, it is noted that valyl esters of ganciclovir and acyclovir (e.g. valganciclovir and valacyclovir, etc.) are believed to have better bioavailibilty in the systemic form (Sugawara et al. (2000) J. Pharm. Sci., Jun, 89(6): 781-789.)

The practice of the invention employs, unless otherwise indicated, conventional techniques of protein chemistry, molecular virology, microbiology, recombinant DNA technology, and pharmacology, which are within the skill of the art. Such techniques are explained fully in the literature (see, e.g., Current Protocols in Molecular Biology, Ausubel et al., eds., John Wiley & Sons, Inc., New York, 1995, and Remington's Pharmaceutical Sciences, 17th ed., Mack Publishing Co., Easton, Pa., 1985.

In still another embodiment, this invention provides kits for the elimination of mammalian cells infected with human papillomavirus according to the methods described herein. In one preferred embodiment, the kits comprise one or more containers containing a nucleic acid construct comprising a nucleic acid encoding a cytotoxin gene under the control of an HPV promoter. The kit may, optionally, comprise one or more cationic lipids and/or liposomes, or other agent suitable for the transfection of the nucleic acid construct. In certain embodiments, the nucleic acid construct may comprise a cream or ointment. The kits may optionally include one or more cell lines for propagation of the vector(s).

The kits may optionally include any reagents and/or apparatus to facilitate the delivery of the molecules described herein. Such reagents include, but are not limited to buffers, pharmacological excipients, labels, labeled antibodies, labeled nucleic acids, filter sets for visualization of fluorescent labels, blotting membranes, and the like.

In addition, the kits may include instructional materials containing directions (i.e., protocols) for the practice of the assay methods or transfection of cells as described herein. Preferred instructional materials provide protocols for reducing or eliminating cells infected with HPV according to the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Virus-Specific Treatment of Human Papillomavirus Type 16-Infected Cells Using the Herpes Simplex Virus 1 Thymidine Kinase Gene

Human papillomaviris type 16 (HPV 16) is associated with development of anogenital squamous cell cancers (SCC) and their precursors, intraepithelial neoplasia (IN). Few approaches to the treatment of intraepithelial neoplasia to prevent SCC are targeted specifically to HPV. We have designed an HPV-specific therapy using the herpes simplex virus-1 thymidine kinase (HSV 1-TK) gene driven by an HPV-specific promoter in the HPV 16 long control region (LCR) (nt 7450-nt 104) which is regulated by the HPV E2 protein. Expression of the HSV 1-TK gene in HPV-infected cells is designed to render the cells sensitive to the prodrugs ganciclovir (GCV) and acyclovir (ACV). To assess the specificity of HPV 16 LCR, we measured the activity of a luciferase expression plasmid under the control of the HPV 16 LCR. A 20-fold induction of luciferase activity was observed in HPV16 E2-expressing cells (CaSki) and a 10-fold induction was seen in HeLa cells expressing HPV18 E2, when compared to HSC3 cells with no expression of E2. Transfection of a plasmid expressing the HSV 1-TK gene driven by the HPV 16 LCR promoter in CaSki cells resulted in approximately 80% growth inhibition when the cells were cultivated in 20-30 μg/ml of either GCV or ACV for 6-8 days and we showed that the cell death was mediated by apoptosis. These results indicate that the direct gene transfer of the HSV 1-TK gene into HPV-positive keratinocytes with TK expression regulated by the HPV E2 protein, accompanied by administration of GCV or ACV, is a clinically feasible therapeutic strategy against HPV-infected cells.

Materials and Methods.

Plasmid Construction.

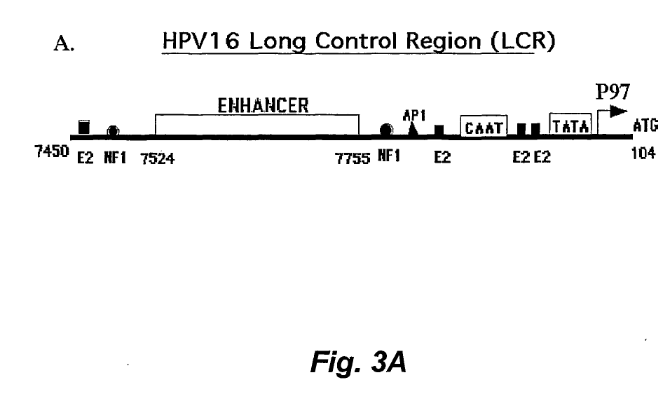

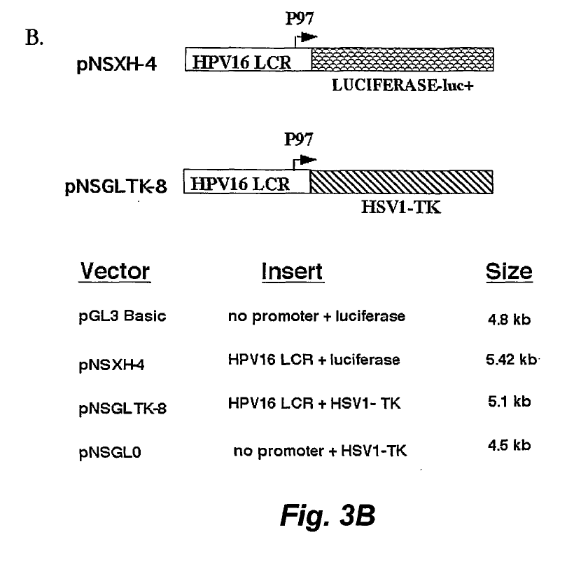

A 620 bp DNA fragment (nt 7420-nt 117) spanning the LCR of the HPV 16 genome shown in FIG. 3A, was PCR-amplified from the plasmid pHPV 16 (obtained from ATCC) using the following primers: Primer URR1: 5′-CGG CTC GAG TGT AGC GCC AGG CCC ATT-3′ (SEQ ID NO:2, the underlined sequence is the cleavage site for XhoI enzyme), and Primer URR2: 5′-CGG AAG CTT GGG TCC TGA AAC ATT GCA-3′ (SEQ ID NO:3, the underlined sequence is the cleavage site for HindIII site). The resulting PCR product shown in FIG. 3A, was sequenced at the UCSF Biomolecular Resource Center to confirm the nucleotide sequence (Seedorf et al. (1985) Virology, 145: 181-185). The sequence contained four copies of the 12-base pair palindromic sequence, ACCN₆GGT (SEQ ID NO:1), which serves as the E2BS. The PCR-amplified LCR fragment was cloned into the XhoI-HindIII site of the pGL3-Basic Vector (Promega, Wis.) upstream of the luc+ cDNA encoding for the modified firefly luciferase (plasmid pNSXH-4).

To construct a plasmid in which the HSV 1-TK gene is driven by the full-length HPV16 LCR (pNSGLTK-8), a 1.3 kb fragment encompassing the HSV 1-TK coding sequence was amplified using PCR from the vector pRB103.

PCR was performed using the following primers: TK-forward: 5′-CGG AAG CTT CCC AGG TCC ACT TCG CAT-3′ (SEQ ID NO:4, the underlined sequence is the cleavage site for HindIII site) and TK-reverse: 5′-CGG TCT AGA CAT AGC GCG GGT TCC TTC-3′ (SEQ ID NO:5, the underlined sequence is the cleavage site for XbaI enzyme). The PCR-amplified product was sequenced at the UCSF Biomolecular Resource Center to confirm the nucleotide sequence. The luciferase gene was excised from the plasmid pNSXH-4 as an approximately 1.7 kb HindIII-XbaI fragment and was replaced by the 1.3 kb PCR product containing HSV 1-TK encoding sequence resulting in the plasmid pNSGLTK-8, in which HSV 1-TK is downstream of the HPV16 LCR promoter element. A control plasmid pNSGL0 containing only HSV 1-TK gene but no HPV16 LCR was constructed by excising the 620 bp XhoI-HindIII LCR sequence from the plasmid pNSGLTK-8. The resulting fragment was filled in with the Klenow fragment and dNTPs and re-ligated to yield pNSGL0. The resulting plasmids are shown in FIG. 3B.

Cell Culture and Transfections.

The HPV 16-positive immortalized CaSki (41) and SiHa (Baker et al. (1987) J. Virol., 61(4): 962-71; el Awady et al. (1987) Virology, 159(2): 389-98) cervical cancer cell lines, the HPV 18-positive HeLa cervical cancer cell line (Boshart et al. (1984) EMBO J., 3(5): 1151-1157), and an immortalized HPV-negative oral cancer cell line, HSC3 (23) were used in this study. All cell lines were grown in Dulbecco's modified Eagle medium (DMEM) supplemented with glutamine (25.0 μg/ml), antibiotics and 10% fetal bovine serum (10% DMEM).

All transfections were performed by calcium phosphate mediated transfection using the Profection Mammalian Transfection Systems (Promega, Wis.) according to the manufacturer's instructions. Cells were seeded at a density of 2×10⁵ cells in each well in two mls of DMEM and grown to 50-60% confluence. One μg of plasmid DNA was used for transfecting each cell line. The calcium phosphate-DNA mix was overlaid on the cells for four to six hours and then replaced by two mls of complete medium. For mock-transfections, no DNA was added to the calcium phosphate transfection reagent. To measure promoter activity with a standard luciferase assay (Promega, Wis.) the transfected cells were harvested after 24-48 hours. To analyze HSV 1-TK gene expression by RT-PCR, the transfected cells were harvested 48-60 hours after transfection.

The plasmid pcDNA3.1/His/lacZ (Invitrogen, Carlsbad, Calif.) was used as a reporter plasmid to assay for the β-galactosidase expression to determine transfection efficiency. The transfected cells were stained with the X-Gal reagent to measure β-galactosidase activity. The transfected cells were washed twice in PBS, fixed in a solution of 2% formaldehyde, 0.2% glutaraldehyde in PBS, pH 7.3 for ten minutes at room temperature. The cells were washed twice in PBS and stained with the X-Gal reagent at 37° C. for 30 minutes to two hours. Cells that had been successfully transfected with the plasmid pcDNA3.1 stained blue when visualized under a light microscope. The number of blue cells was counted to determine transfection efficiency.

Detection of Gene Expression by RT-PCR.

Poly (A+) messenger RNA (mRNA) was prepared from the cell lines using a mRNA isolation kit (Boehringer Mannheim, Ind.). Approximately 2×10⁶ cells were harvested for RNA isolation. After washing twice in ice-cold PBS, cells were lysed in a lysis buffer and mechanically sheared by passing cells through a 21-gauge needle. Fifty pmol of biotin-labeled oligo(dT)₂₀ were added to the lysate and then mixed with streptavidin magnetic particles for five minutes at 37° C. The magnetic particles were washed three times in a washing buffer. The mRNA was eluted by incubation at 65° C. for two minutes, separated completely from the magnetic particles and quantitated by measuring the O.D. at A₂₆₀.

To confirm expression of the HSV 1-TK gene, RT-PCR was performed using gene-specific primers NSTK1, 5′-CGT TCT GGC TCC TCA TGT CG-3′ (SEQ ID NO:6) and NSIK-2, 5′-GCC AGC ATA GCC AGG TCA AG-3′ (SEQ ID NO:7), which amplify a 288-bp region of the TK gene. Expression of the HPV16 E2 gene was analyzed by RT-PCR using gene-specific primers NSE2-4, 5′-GTA TGG GAA GTT CAT-3′ (SEQ ID NO:8) and NSE2-5, 5′-CTT AGT GGT GTG GCA G-3′ (SEQ ID NO:9) which amplify a 216-bp fragment (spanning nt 3300 to nt 3516) in the coding sequence of HPV16 E2 gene. RT-PCR was performed using the Superscript One-Step RT-PCR system (Life Technologies, Rockville, Md.). 0.2 μg of mRNA was used for a first-strand cDNA synthesis at 50° C. for 30 minutes and denaturation at 94° C. for two minutes, followed by 35 cycles of PCR amplification consisting of: 94° C. for 15 seconds, 55° C. for 30 seconds and 72° C. for one minute and a final extension at 72° C. for ten minutes. The resulting PCR products were analyzed on a 1.2% agarose gel by standard gel-electrophoresis.

Detection of Luciferase Expression by the Luciferase Assay.

The CaSki, HSC3 and HeLa cell lines were transfected with the plasmid pNSXH-4 and the control basic plasmid pGL3 as described above. After 24 hours the cell lysates were harvested with 50.0 μL luciferase assay buffer (Promega, Wis.) and centrifuged to remove debris. The lysates were combined with luciferin reagent to measure luciferase reporter gene expression using the Luciferase Assay System (Promega, Wis.) according to the manufacturer's instructions. The cell lysates were serially diluted and luciferase expression was measured as relative light units (RLU) using a Dynatech Microlite Plate Luminometer with signal integration for ten seconds.

In vitro Effects of GCV and ACV on HSV-TK+ Cell Lines.

The CaSki, SiHa, HSC3 and HeLa cell lines were transfected with the pNSGLTK-8 or pNSGL0 plasmids or mock transfected with no plasmid. Gene expression was allowed for 48 hours after transfection, after which the transfected cells were exposed to either GCV (Cytovene, Roche, Calif.) or ACV (Novapharm, USA Inc, Ill.). GCV or ACV killing curves were determined for the transfected cell lines by culturing the cells in different concentrations of the two drugs ranging from 040.0 μg/ml final concentration in 10% DMEM. Growth was allowed to proceed in the presence of GCV or ACV for 6-10 days, after which the cell viability was measured by the MTS cell proliferation assay (Berridge and Tan (1993) Arch. Biochem. Biophys. 303: 474) (CellTiter 96 AQᵤₑᵤₒᵤₛ One Solution Cell Proliferation Assay, Promega, Wis.), according to the manufacturer's instructions. The absorbance at 490 nm is a measure of the number of living cells in the culture. Percent survival was calculated as follows:

$$\text{\% survival} = \frac{{\text{Absorbance}}_{490} \text{test} - {\text{Absorbance}}_{490} \text{background}}{{\text{Absorbance}}_{490} \text{untreated cells} - {\text{Absorbance}}_{490} \text{background}} \times 100$$

Detection of Apoptosis in Transfected Cell Lines.

The CaSki, SiHa, HSC3 and HeLa cell lines were transfected with the pNSGLTK-8 or pNSGL0 plasmids or mock transfected with no plasmid. TK gene expression was allowed for 48 hours after transfection, after which the transfected cells were cultured in different concentrations of either GCV or ACV ranging from 0-40.0 μg/ml final concentration in 10% DMEM over a period of six to ten days. The DeadEnd Colorimetric Apoptosis Detection System (Promega, Wis.) is a modified TUNEL (TdT-mediated dUTP Nick-End Labeling) assay in which the enzyme Terminal deoxynucleotidyl Transferase (TdT) incorporates biotinylated nucleotides at the 3′-OH DNA ends (1, 20). Streptavidin horseradish-peroxidase is then bound to the biotinylated nucleotides, which are detected by hydrogen peroxide and diaminobenzidine (DAB), a stable chromogen. Using this procedure, apoptotic nuclei stained dark brown when visualized under a light microscope.

Results.

Upregulation of Luciferase Reporter Expression from a Vector Containing the HPV 16 LCR in Cell Lines Expressing the HPV E2 Protein.

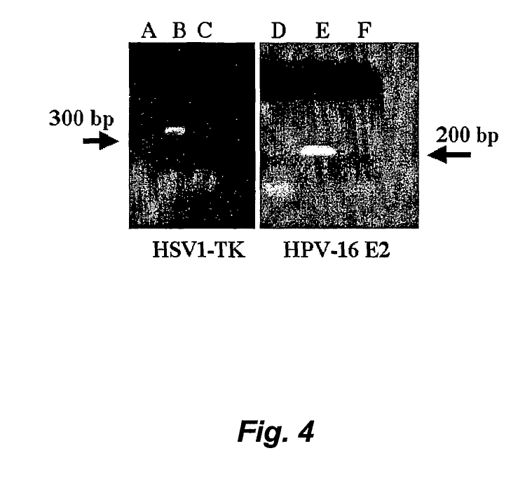

To determine if the HPV 16 LCR promoter was sufficient to drive the expression of exogenous genes in HPV 16-positive cell lines, we transfected CaSki cells with plasmid pNSXH-4 and as a control, the pGL3 Basic plasmid (Promega, Wis.) upstream of the luciferase reporter gene. RT-PCR analysis of the CaSki cell line using E2-specific primers showed that the cell line did express the E2 gene whereas the HPV-negative oral epithelial HSC3 cell line did not show E2 expression (FIG. 4).

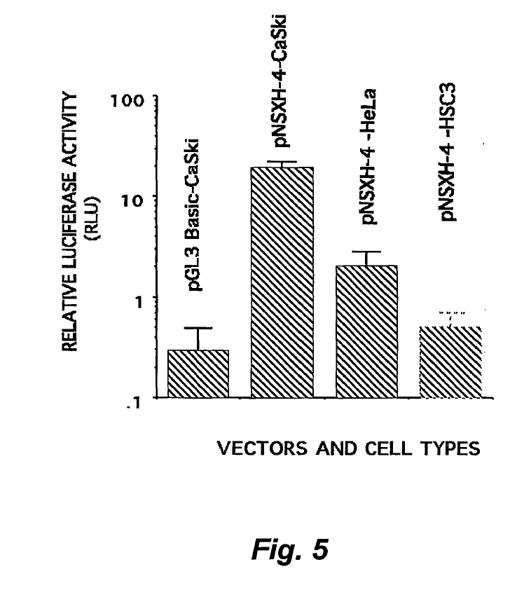

The results in FIG. 5 show that CaSki cells transfected with pNSXH-4 expressed more than 20-fold higher luciferase activity when compared to CaSki cells transfected with pGL3 containing no LCR. In contrast, when the plasmid pNSXH-4 was transfected into HPV-negative HSC3 cells, the level of luciferase activity was comparable to the level of expression with the control parental plasmid pGL3.

The transactivation domains of E2 proteins from HPV 16 and from HPV 18 are functionally homologous (22). To determine the specificity of this plasmid for upregulation by the HPV 16 E2 protein for comparison with that of HPV 18, we transfected the pNSXH-4 and pGL3 plasmids into the HPV 18-positive HeLa cell line. The results in FIG. 5 show that pNSXH-4 is activated in HeLa cells, approximately 12-fold higher than the level of luciferase expression detected with pGL3 control plasmid but that this expression level is approximately half of that measured for HPV 16-positive CaSki cells. Together, these data show that the HPV 16 LCR can be transactivated by the HPV 16 E2, and to a lesser extent by the HPV 18 E2 protein to drive the expression of an exogenous gene.

HSV 1-TK Gene Expression Driven by the HPV 16 LCR Results in GCV and ACV Sensitization.

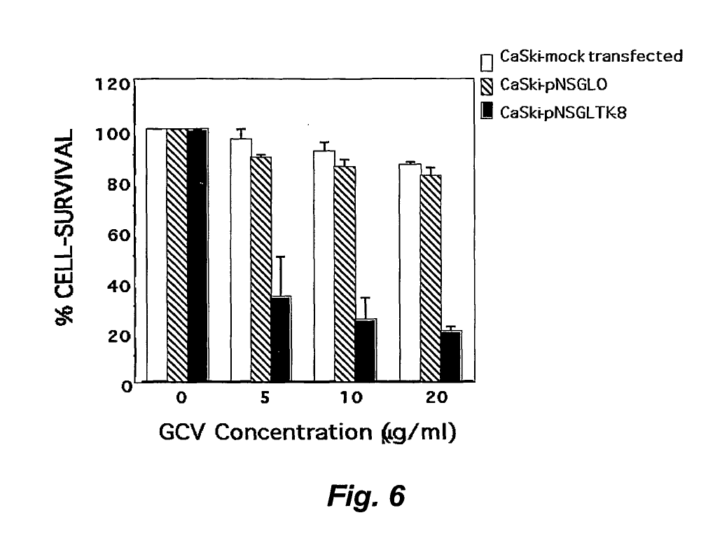

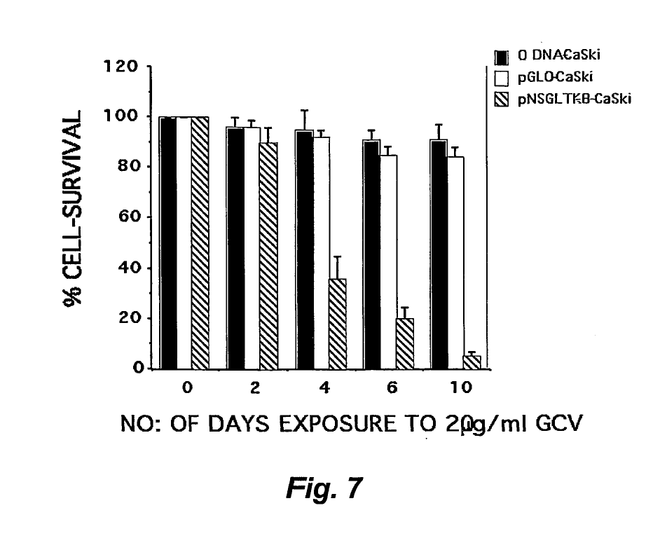

Expression of the HSV 1-TK gene in the transfected CaSki cells was confirmed by RT-PCR as shown in FIG. 4. HSV 1-TK mRNA was detected in CaSki cells 7 days post-transfection, whereas no expression of HSV 1-TK gene was detected in HSC3 cells, which also did not exhibit any E2 gene expression. To determine if expression of HSV 1-TK under the control of the HPV 16 LCR conferred susceptibility to GCV or ACV, we compared GCV and ACV sensitivity of cells transfected with the pNSGLTK-8 plasmid with CaSki cells that were not transfected and with cells that were transfected with the pNSGL0 control plasmid (FIG. 3B). CaSki cells were transfected and exposed to increasing concentrations of either GCV or ACV as described previously. FIG. 6 shows that there was a dose-dependent effect of GCV on cell viability in cells transfected with the plasmid pNSGLTK-8 and expressing the HSV 1-TK gene. Cell viability decreased with increasing concentrations of GCV with maximal (approximately 80%) cell death observed at a concentration of 20.0 μg/ml of GCV after six days of exposure to the prodrug (FIG. 6). No significant effect on cell viability was observed on CaSki cells when they were either mock-transfected or transfected with the control plasmid pNSGL0. Treatment of CaSki cells transfected with pNSGLTK-8 with 20.0 μg/ml GCV for 10 days killed more than 95% cells in culture (FIG. 7) showing progressive cell death.

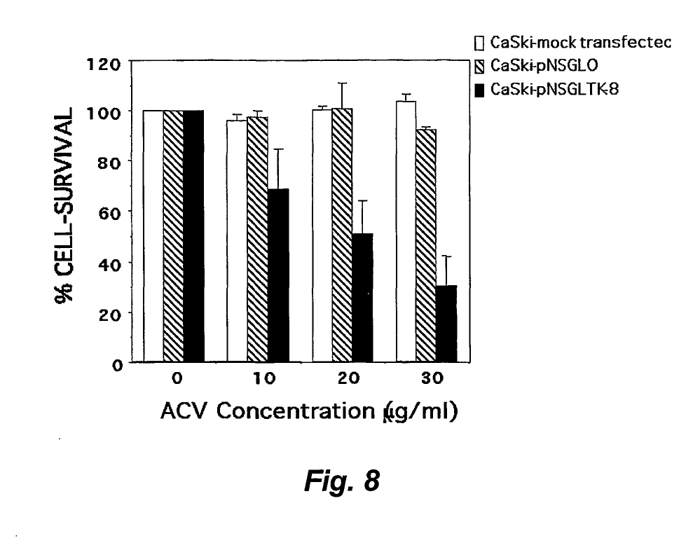

Sensitization of CaSki cells transfected with pNSGLTK-8 was also tested after exposure to different concentrations of ACV (FIG. 8). ACV showed cytotoxic effects at a higher concentration of 30.0 μg/ml than GCV and after a longer period of exposure than GCV (ten days) (FIG. 8). CaSki cells that were mock-transfected or that were transfected with the pNSGL0 plasmid were refractory to ACV. These results show that only cells expressing HSV 1-TK gene become sensitive to the prodrugs. GCV or ACV at the concentrations tested have no toxicity for mock-transfected cells or cells that do not express HSV 1-TK.

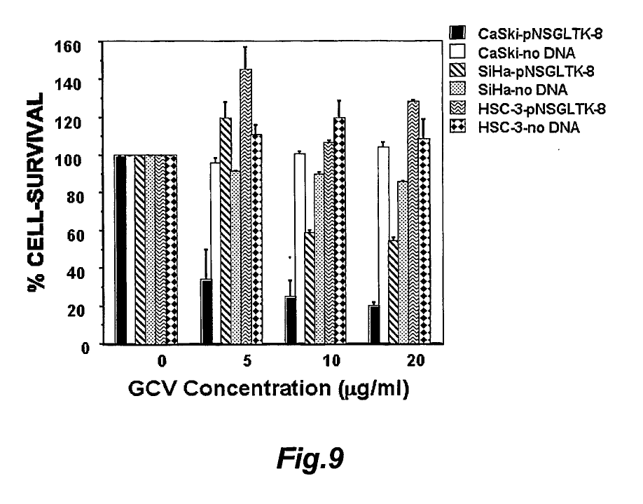

To determine whether GCV-mediated killing was specific to TK-transfected CaSki or SiHa cells, we also tested the cytotoxic effects of GCV in the SiHa HPV 16-positive cervical cancer cell line and HSC3 cells. The results are shown in FIG. 9. These data show that the SiHa cell line was sensitized to GCV only when transfected with pNSGLTK-8 with approximately 42% of SiHa cells killed with GCV treatment after six days. With prolonged exposure for two weeks with 20.0 μg/ml GCV, more than 90% of pNSGLTK-8 transfected SiHa cells and HeLa cells were killed (data not shown).

To determine if the lower toxicity noted with SiHa cells than CaSki cells may have reflected differences in transfection efficiency, we performed control transfections using the β-galactosidase expression plasmid. These experiments showed that the transfection efficiency for the calcium phosphate transfection method was similar for all cell lines tested, i.e., 40%-60% (data not shown). In all of the mock-transfected cell lines tested, cell viability was not affected by the exposure to the DNA-calcium phosphate transfection complex or due to the cytotoxic effects of the different concentrations of the prodrugs used (FIG. 9).

GCV and ACV Treatment of Cells Expressing HSV 1-TK Induces Apoptosis.

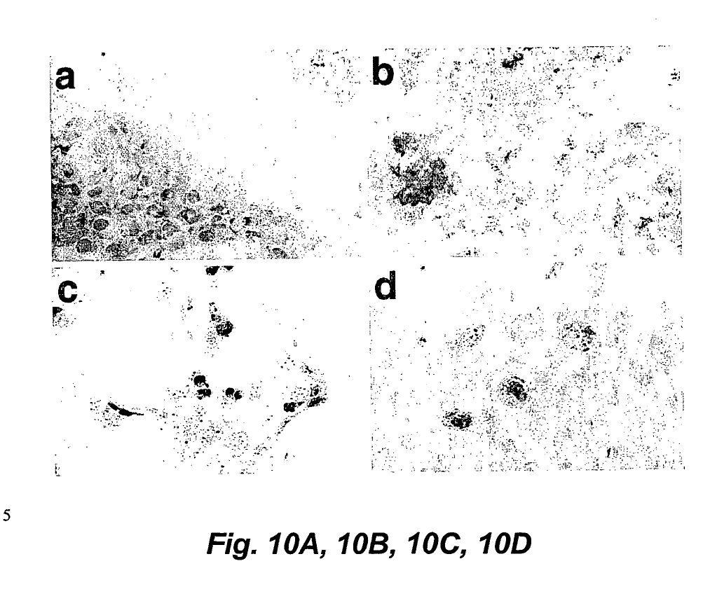

A number of studies have reported that GCV treatment induces apoptosis in cells expressing HSV 1-TK (Elshami et al. (1996) Gene Therapy. 3: 85-92; Hamel et al. (1996) Cancer Res., 56: 2697-2702) as shown by oligonucleosomal DNA-laddering, nuclear morphological aberrations and endonucleolytic activity using TdT assays (Arends et al. (1990) Amer. J Path. 136:593; Gorczyca et al (1993) Cancer Res., 53: 1945-1951). Apoptotic cells have been shown to fragment into apoptotic bodies, which may be phagocytosed and digested by macrophages, or neighboring cells. To determine if CaSki and SiHa cells expressing HSV-1 TK were undergoing apoptosis in the presence of GCV, we examined the morphology of cells after treatment with 20.0 μg/ml GCV. CaSki and SiHa cell lines transfected with the plasmid pNSGLTK-8 were cultured in the presence of different concentrations of GCV ranging from 0-20.0 μg/ml final concentration for a period of seven days and examined by TUNEL staining. After incubation of the pNSGLTK-8 transfected CaSki cells in 20.0 μg/ml GCV for four days, the cells began to show characteristic apoptotic morphology (FIG. 10). Microscopic evaluation revealed that the nuclei appeared highly condensed and fragmented after four days of treatment (FIG. 10), and after six days of GCV treatment, TUNEL staining revealed highly fragmented chromosomal DNA which stain dark brown. The cytoplasm of apoptotic cells also had a distinct punched-out appearance and in a large number of cells, membrane blebbing was also observed. In addition, by six days of GCV treatment very few cells were visible in the field because of cell death.

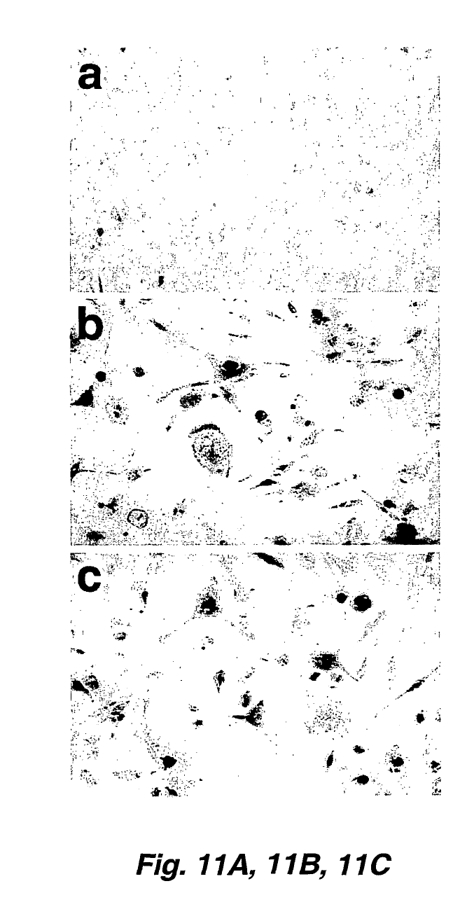

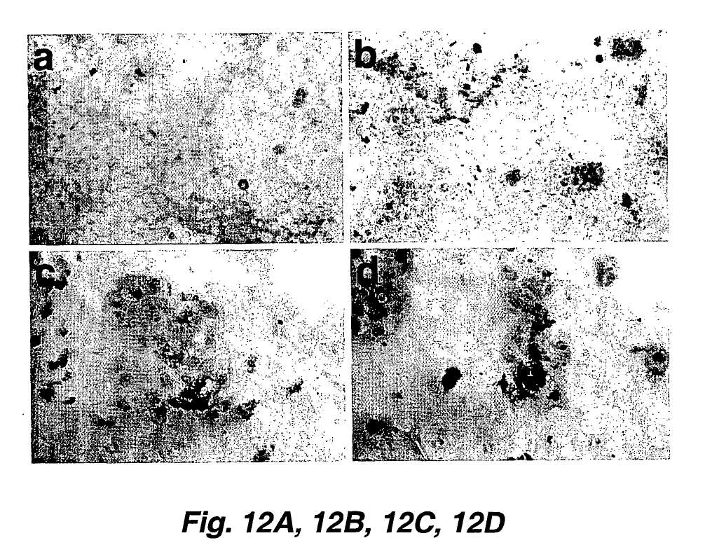

SiHa cells also showed significant cell death associated with typical apoptotic morphology after transfection with the plasmid pNSGLTK-8 and treatment with 20.0 μg/ml GCV for eight to ten days (FIG. 11), as did cells treated with 20.0 μg/ml of ACV (FIG. 12). Typical apoptotic morphology of TUNEL-stained nuclei was observed six days after ACV exposure with very pronounced effect seen after ten days of treatment (FIG. 12). In the absence of either GCV or ACV, only basal levels of apoptosis was observed. TUNEL staining of untransfected cell lines treated with 20.0 μg/ml concentrations of either GCV or ACV for six to eight days did not show an increase in apoptotic cells (data not shown).

In addition, in a phenomenon known as the “bystander effect”, cells that do not express TK may be killed in the presence of cells that do express HSV 1-TK (Chen et al. (1995) Human Gene Therapy. 6: 1467-1476; Elshami et al. (1996) Gene Therapy. 3: 85-92; Fick et al. (1995) Proc. Natl. Acad. Sci., USA, 92: 11071-11075; Freeman et al (1993) Cancer Res., 46: 5276-5281; Hamel et al. (1996) Cancer Res., 56: 2697-2702). This could occur by either transfer of GCV-laden cellular and nuclear material between cells or by phagocytosis of apoptotic bodies. In HSV 1 TK-transfected cells, apoptotic bodies can be seen containing nuclear fragments or cellular organelles, consistent with phagocytosis (FIG. 10C(c), 9C(d)).

Discussion.

Treatment of HPV-associated high-grade CIN has been shown to reduce the incidence of cervical SCC and treatment of high-grade anal IN may similarly reduce the incidence of anal SCC. Current treatment modalities are not HPV-specific and rely primarily on physical destruction or removal of the lesion. These methods may be painful and expensive, especially in the anal canal. Lesion recurrence is not uncommon in either the cervix or anus and mandates continued close follow-up of patients after therapy. Treatments targeted specifically to HPV-infected cells would represent an important therapeutic advance and could potentially be used alone or in conjunction with current therapeutic approaches. Some high-risk individuals, notably those with HIV-associated immunosuppression do not respond as well to routine therapy and often require multiple course of treatment with different modalities (Kiviat et al. (1993) AIDS. 7: 4349; Maiman et al. (1993) Obstet Gynecol. 82: 170-174; Maiman (1998) J Natl Cancer Inst Monogr. 23: 4349; Palefsky (1994) AIDS 8: 238-295; Palefsky (1995) Current Opinions in Oncology, 7: 437441; Palefsky (1998) J. National Cancer Institute. Monographs, 23:15-20; Palefsky et al. (1998) J Acquir Immune Defic Syndr Hum Retrovirol. 17(4): 320-326; Palefsky et al. (1998) AIDS 12(5): 495-503). Newer approaches to therapy that are in early stages of investigation include immune modulation through therapeutic vaccination against HPV 16 proteins or modulation of local immune response with drugs such as imiquimod. However, an HPV-specific therapeutic approach, such as that described in this report might be of particular value in HIV-positive individuals since it does not require an intact immune response.

To assess the feasibility of this HPV-specific gene therapy approach, we designed a system to transfer to HPV-infected cells the HSV 1-TK suicide gene under the control of HPV E2-responsive elements. We have shown that the expression of HSV 1-TK renders HPV 16-positive cells sensitive to nontoxic prodrugs such as GCV or ACV. Currently, the HSV 1-TK/GCV approach is under investigation in clinical trials for several other diseases (Marcel and Grausz (1997) Gene Therapy enrolement report, end 1996. Human Gene Therapy, 8: 775-800; Takamiya et al. (1992) J. Neuroscience Res. 33: 493-503; Tong et al. (1996) Gynecologic Oncology. 61 (2), 175-179; Yee et al. (1996) Human Gene Therapy. 7(10), 1251-1257). The HSV 1-TK/GCV strategy has been used to efficiently treat solid tumors generated from mammary epithelial tissue in rats (Wei et al. (1998) Cancer Res., 58(16): 3529-3532). Transcriptionally activated tumor- or tissue-specific suicide gene therapy approaches have also been successfully used for pituitary lactotrophic cells (Southgate et al. (2000) Endocrinology, 141(9): 3493-505) and for breast cancer cells (Pandha et al. (1999) J. Clinical Oncology, 17(7): 2180-2189). A successful phase I clinical trial was conducted to test the safety and efficacy of a breast cancer-specific genetic prodrug activation therapy targeted by the use of human erbB-2 gene promoter (Pandha et al. (1999) J. Clinical Oncology, 17(7): 2180-2189). This approach was shown to be safe and resulted in targeted gene expression in up to 90% cases. These studies, along with our results in this study, are encouraging for the development of genetic prodrug activation therapies that exploit the unique transcriptional profile of tumor- or virus-infected cells.

For the treatment of anogenital IN, we propose an approach consisting of transfer of the HSV 1-TK suicide gene to HPV 16-infected anogenital mucosal epithelium with expression under the control of the HPV 16 LCR, combined with an oral course of GCV, ACV or other active oral drugs such as valganciclovir. Since these lesions are mucosal and relatively accessible, it might be feasible to deliver effective levels of plasmid using a topical approach with a liposomal delivery. A number of liposomal/DNA formulations have been successfully tested in preclinical and clinical studies where cationic liposomes serve as carriers of DNA to defined regions, e.g., lung, nasal epithelium, arterial endothelium, spleen, brain and a number of tumors (Hyde et al. (2000) Gene Therapy, 7, 1156-1165; Reimer et al. (1999) J. Pharmacol. Experimental Therapeutics, 289(2): 807-815; Takakuwa et al. (1997) Japanese Journal Cancer Res., 88(2): 166-175). These studies have shown that DNA lipoplexes are effective in gene delivery where topical or localized application is an appropriate route of administration. A recent gene-therapy study has successfully performed repeat administrations of DNA/liposomes to the nasal epithelium of patients with cystic fibrosis with substantial efficacy (Hyde et al. (2000) Gene Therapy, 7, 1156-1165).

Our approach exploits transactivation of the HPV LCR by the E2 protein and the ability of the HPV (e.g. HPV 16) LCR to drive the expression of the exogenous gene(s). Therefore we selected-cell lines with HPV (e.g. HPV 16) genomic sequences that can provide E2 protein in trans. The gene products of the E2 ORF possess trans-acting functions targeting enhancer sequences and E2-responsive sequences located in the LCR (Bernard and (1994) Arch. Dermatalogy. 130: 210-215; Bouvard et al. (1994) EMBO J. 13: 5451-5459; Broker et al. (1989) Cancer Cells. Molecular Diagnostics of Human Cancer by Cold Spring Harbor Laboratory, 197-208; Cripe et al. (1987) EMBO J. 6: 3745; McBride et al. (1991) J. Biol Chem. 266: 18411-18414; Schwarz et al. (1985) Nature 314: 111). Our experiments were done in the CaSki cell line that has more than 250 copies of HPV 16 genomes and in the SiHa cell line that has one to two copies of HPV genomic DNA (Meissner (1999) Journal of General Virology. 80: 1725-1733). Expression of E2 was confirmed in both cell lines and both cell lines were killed after transfection with HSV 1-TK and exposure to GCV. Killing was observed to occur faster in the CaSki cells than the SiHa cells, and this may reflect the higher HPV 16 DNA copy number and possibly higher levels of E2 protein expression. Cells that were mock-transfected or transfected with a control plasmid did not show toxicity, nor did HPV-negative cells. Together, our results show that the HPV 16 LCR can be activated by E2 protein to drive the expression of the HSV-1 TK gene and render the cells susceptible to GCV or ACV.

In addition to the role of the E2 protein in our system, E1 expression may also be important. The E1 protein in association with E2 protein from HPV-positive cells may interact with ori, the viral sequence for origin of replication, to induce plasmid DNA replication (Gadi et al. (1999) American Journal of Resp. Cell and Mol. Biol. 20: 1001-1006). This can be used to advantage in the design of this gene therapy strategy. The high level of luciferase activity in transfected CaSki cells may reflect plasmid replication since the LCR sequences included in our vector contained ori sequences whereas pGL3 basic plasmid has no origin of replication in mammalian cells.

Our data suggest that this approach should be specific to HPV-infected cells, with the exception of the “bystander effect”. It has been previously observed that GCV treatment of cells expressing HSV 1-TK kills more cells than are actually expressing the HSV 1-TK gene. A study using the HSV 1-TK/GCV strategy on localized tumors resulting from mouse colon carcinoma cells showed that although only 10%-20% of the tumor cells were transfected with the HSV 1-TK gene, the tumors showed almost complete regression due to the “bystander effect” (Gagandeep et al. (1996) Cancer Gene Therapy, 3(2): 83-88). Although the mechanisms involved in the bystander effect are not completely understood, it is possible that it is caused by intercellular transfer of active phosphorylated GCV or ACV from infected cells to uninfected neighboring cells. The bystander effect can partially occur through cell-cell contact and intercellular communications, or gap junctions (Chen et al. (1995) Human Gene Therapy. 6: 1467-1476; Elshami et al. (1996) Gene Therapy. 3: 85-92; Fick et al. (1995) Proc. Natl. Acad. Sci., USA, 92: 11071-11075; Freeman et al. (1993) Cancer Res., 46: 5276-5281 21) through which GCV-phosphate can circulate between TK-positive and TK-negative cells. Phagocytosis of GCV-phosphate laden debris by adjacent cells can also lead to cell death (Hamel et al. (1996) Cancer Res., 56: 2697-2702) and we have observed such phagocytosis in transfected HPV 16 cell lines.

A number of studies have shown that GCV-induced cell death along with the bystander effect is mediated by apoptosis (Hamel et al. (1996) Cancer Res., 56: 2697-2702). In our study we similarly observed cellular morphology and DNA fragmentation characteristic of apoptosis. Since CaSki and SiHa are HPV 16-positive cell lines in which the p53 protein is depleted or is expressed at a very low level (Scheffner et al. (1990) Cell, 63(6): 1129-1136; Thomas et al. (1999) Oncogene 18, 7690-7700), it is possible that GCV-induced apoptosis in these cell lines is occurring through a p53-independent mechanism.

In models of HPV-induced carcinogenesis in vivo, it has been shown HPV infection is initially established in basal and parabasal layers of the epithelium (Broker et al. (1989) Cancer Cells. Molecular Diagnostics of Human Cancer by Cold Spring Harbor Laboratory, 197-208; Stoler et al. (1992) Human Pathology, 23(2): 117-128). These cells constitute a potentially long-term reservoir of HPV infection. Although the basal cells contain small numbers of episomal copies of HPV and express low levels of viral proteins such as E2, viral replication and protein expression increase as the cells differentiate. At low levels, the E2 protein upregulates activity of the E6 promoter in the LCR but as the E2 protein accumulates, the E6 promoter is repressed (Broker et al. (1989) Cancer Cells. Molecular Diagnostics of Human Cancer by Cold Spring Harbor Laboratory, 197-208; Schwarz et al. (1985) Nature 314:111; Stoler et al. (1992) Human Pathology, 23(2): 117-12847). A strategy that relies on E2 expression to upregulate expression of an exogenous gene under the control of the E6 promoter would therefore be expected to have maximal toxicity in cells expressing low levels of E2. Therefore, we believe our approach is especially well suited to elimination of HPV-infected basal and parabasal cells, and consequently expected to be effective in eliminating the reservoir of HPV infection. The anogenital epithelium is continuously regenerated by the basal and parabasal cell layers. If HPV-negative basal and parabasal cells replace the HPV-positive cells that are eliminated, over time the regenerating epithelium will become progressively free of HPV infection. This therapeutic approach would therefore involve repeated exposure of the lesion over the course of at least one cycle of epithelial turnover.

Although the approach described here may work well for IN, it may be less effective for treatment of invasive cancer. Integration of the HPV genome into the host chromosomal DNA is often seen in high-grade IN and cancers. With integration the episomal HPV DNA breaks at E1/E2 boundary, disrupting the E1 and the E2 open reading frames (Broker et al. (1989) Cancer Cells. Molecular Diagnostics of Human Cancer by Cold Spring Harbor Laboratory, 197-208; Cripe et al. (1987) EMBO J. 6: 3745; E1 Awady et al. (1987) Virology, 159(2): 389-398; zur Hausen (1991) Virology. 184: 9-13). Loss of E2 expression may attenuate expression of the HSV 1-TK gene and the cancer cells may remain resistant to GCV. Conversely, some cervical cancers express low levels of E2 and these tumors may be at least partially treatable with this approach.

The bystander effect may also kill cells adjacent to cells expressing HSV 1-TK, including some normal uninfected adjacent cells. This effect may also eliminate cells that are more differentiated in the lesion that express higher levels of E2 and which would be expected to have little or no expression HSV 1-TYK Therefore, the ‘innocent bystander effect’ is potentially beneficial in the therapeutic approach described here because it may allow for killing of cells when only a fraction of the cells in a lesion are transfected (Chen et al. (1995) Human Gene Therapy. 6: 1467-14761 Freeman et al. (1993) Cancer Res., 46: 5276-5281; Gagandeep et al. (1996) Cancer Gene Therapy, 3(2): 83-88). It could, however, also lead to toxicity of normal adjacent HPV-negative cells, although this effect would likely be restricted only to cells in the immediate vicinity of the lesion.

It was also noteworthy that the concentrations of GCV and ACV used in this study to induce cell killing were comparable to those demonstrated in other systems (Calvez et al. (1996) Clin. Cancer Res., 2(1): 47-51; Chen et al. (1995) Human Gene Therapy. 6: 1467-1476; 34, 51) and that these concentrations were within the range achievable with oral administration of these drugs. However, in our experiments, we observed more effective killing with GCV than with ACV, which required a higher concentration and a longer exposure to HSV 1-TK positive cells than with GCV. Studies on metabolism of these drugs have shown that ACV is poorly phosphorylated to its active triphosphate form with low DNA incorporation (44). GCV has been shown to produce superior cytotoxicity and induces a multi-log killing through a unique delayed mechanism that allows cells to complete one cell division after which they permanently arrest in early S-phase (44). This suggests that GCV and its recently developed analogs with higher oral bioavailability such as valganciclovir, may be more potent prodrugs for in vivo treatment.

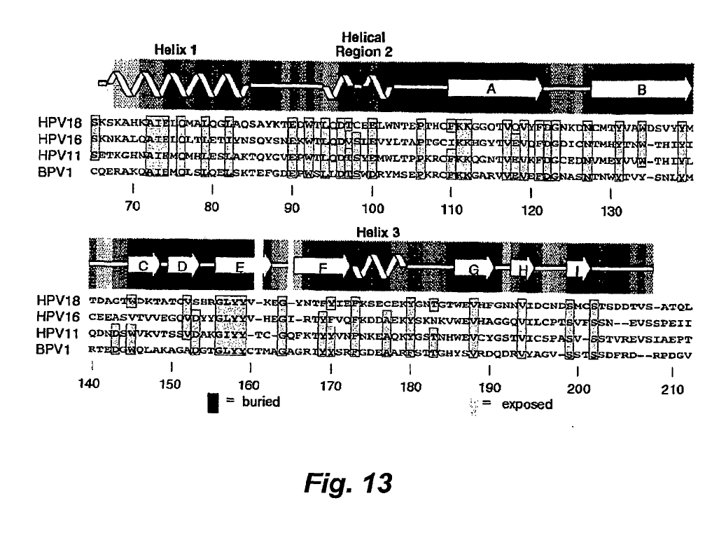

Sequence alignment (FIG. 13) of four papillomavinis E2 proteins, HPV 16, HPV 18, HPV 11 and bovine papillomaviris (HPV)-1 reveals that they have 30% amino acid sequence identity (Harris and Botchan (1999) Science 284 (5420): 1673). These proteins as well as their E1 proteins can functionally complement each other in intertypic cross-variant studies (Harris and Botchan (1999) Science 284 (5420): 1673). In our study, cell killing was also observed in the HPV 18-positive HeLa cell line, indicating that the HPV 16 promoter elements used in our construct were not completely specific to HPV 16. Although use of a plasmid containing HPV 16 promoter elements might be expected to have some efficacy in lesions associated with other HPV types, a type-specific approach to therapy using plasmids containing control elements of the appropriate HPV type may be more efficacious for those lesions. For example, IN caused by HPV 18 could be treated with HPV 18-specific constructs and condylomata acuminata associated with HPV 6 or HPV 11 could be treated with HPV 6- and HPV 11-specific LCR constructs.

In summary, we have shown in vitro evidence for the feasibility of an HPV-specific gene therapy approach consisting of transfection of HSV 1-TK under the control of HPV promoter elements followed by administration of GCV or ACV. Further experiments can be performed to optimize gene delivery methods to the anogenital epithelium as well as testing for safety and efficacy in animal models.

### Example 2

### Specificity of the Cytotoxic Constructs

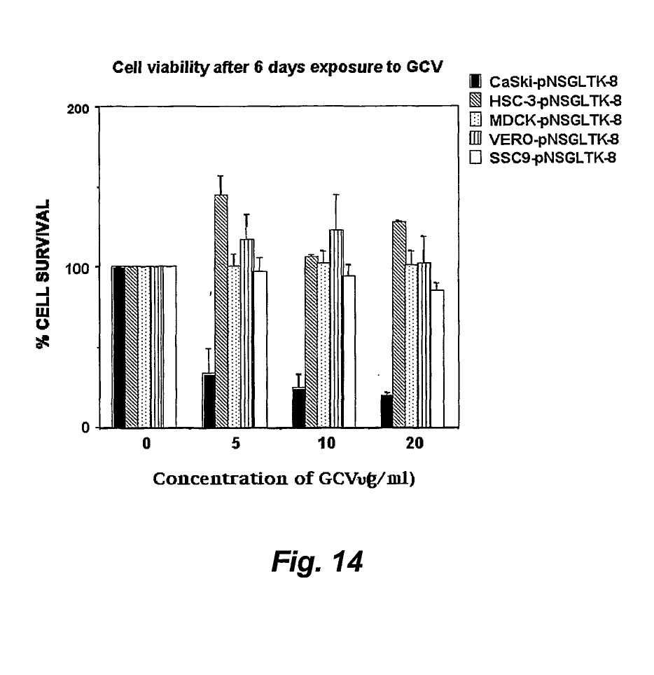

To test the specificity of the methods described herein, an HPV positive cell line (CaSKi) and four HPV-negative cell lines were transfected with the plasmid plasmid pNSGLTK-8 described herein. As shown in FIG. 14, after 6 days exposure to ganciclovir cytotoxicity is restricted to HPV positive cell line CaSKi only. The four other non-HPV cell lines (HSC3—a human oral cancer cell line, MDCK—Madin-Darby canine kidney cell line, VERO—African Green Monkey kidney cell line, and Human oral squamous cell carcinoma cell, SSC9) tested with different concentrations of GCV after being transfected with the plasmid pNSGLTK-8 did not show any significant cytotoxic effect.

### Example 3

### Detection of the Presence HSV1-TK Protein in Apoptotic Transfected Cells

CaSki cells were transfected with HPV16 LCR driven HSV1-TK construct (pNSGLTK-8). After 48 hours, cell were fixed and HSV-TK protein was detected using polyclonal rabbit anti-HSV1-TK antibody and FITC-conjugated goat anti-rabbit secondary antibody. The nuclear staining was done with propidium iodide. The cells were examined by confocal microscopy.

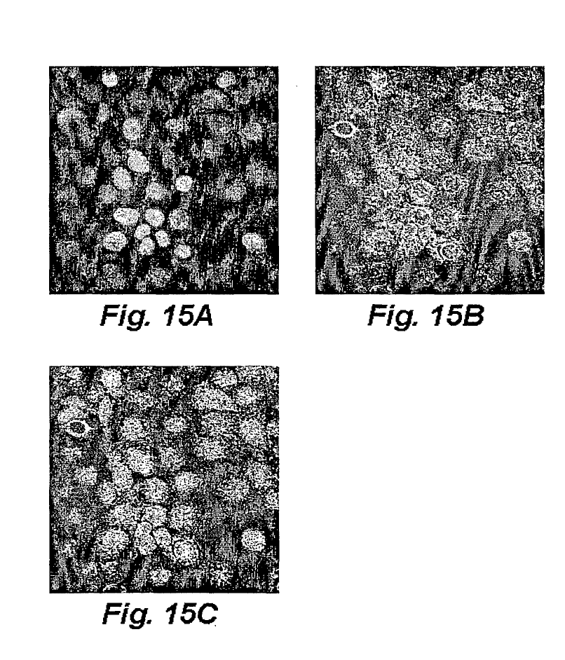

FIG. 15A shows nuclear staining of cells by PI. FIG. 15B shows anti-HSV1-TK staining. FIG. 15C, the merge, shows nuclear and cytoplasmic distribution of HSV1-TK protein with nuclear colocalization shown as yellow (bright “white” in black and white photograph).

In a ganciclovir experiment, CaSki cells were transfected with HPV16 LCR driven HSV1-TK construct (pNSGLTK-8). The cells were treated with 20 ug/ml GCV for 2 days. Cell were fixed and HSV1-TK protein was detected using polyclonal rabbit anti-HSV1-TK antibody and FITC-conjugated goat anti-rabbit secondary antibody. The nuclear staining was done with propidium iodide. The cells were examined by confocal microscopy.

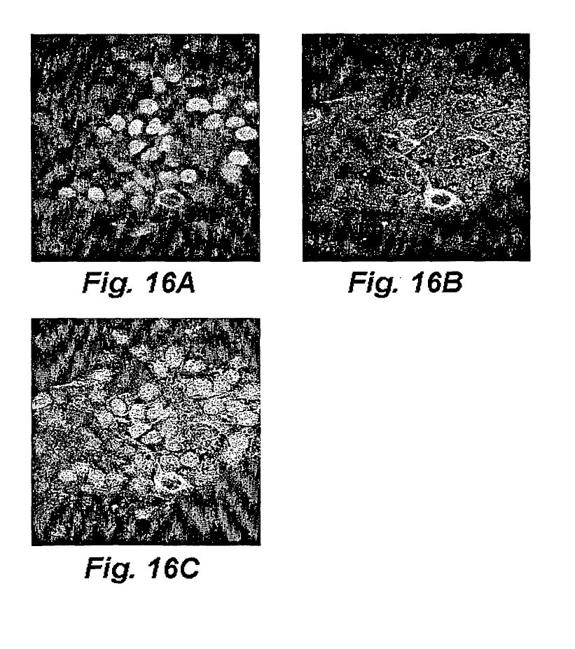

FIG. 16A shows nuclear staining of cells by PI. The majority of nuclei look disrupted due to early apoptosis. FIG. 16B shows anti-HSV1-TK staining. The merge (FIG. 16C) shows nuclear and cytoplasmic distribution of HSV1-TK protein with nuclear colocalization shown as yellow (bright “white” in black and white photograph).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of selectively killing a cell bearing a human papillomaviris (HPV), said method comprising:
transfecting a mammalian cell with a nucleic acid construct encoding an HPV specific promoter that is induced by an HPV protein wherein said promoter is operably linked to a nucleic acid comprising a cytotoxic gene whereby said cell, when infected with a human papilloma virus, induces expression of said cytotoxic gene thereby resulting in the death of said mammalian cell.

2. The method of claim 1, wherein said transfecting comprises delivery using a vector selected from the group consisting of a retroviral vector, an adeno-associated vector (AAV), an adenoviral vector, a herpes viral vector, and a Sindbis viral vector.

3. The method of claim 1, wherein said transfecting comprises using a delivery agent selected from the group consisting of a lipid, a liposome, a cationic lipid, and a dendrimer.

4. The method of claim 1, wherein said promoter is a promoter up-regulated by the HPV E2 protein.

5. The method of claim 1, wherein said promoter is a promoter from HPV-16.

6. The method of claim 1, wherein said promoter comprises a full-length HPV LCR.

7. The method of claim 1, wherein said promoter comprises sufficient length of an HPV LCR to induce transcription of a nucleic acid in response to an E2 protein.

8. The method of claims 6 or 7, wherein said LCR is an HPV-16 LCR.

9. The method of claim 1, wherein said promoter is an HPV16 LCR promoter.

10. The method of claim 1, wherein said cytotoxic gene is selected from the group consisting of a ricin gene, an abrin gene, a pseudomonas exotoxin gene, a diphtheria toxin gene, and a thymidine kinase gene.

11. The method of claim 10, wherein said nucleic acid construct comprises an HPV-16 promoter operably linked to a herpes simplex thymimdine kinase gene.

12. The method of claim 11, wherein said method further comprises contacting said cell with ganciclovir (GCV) or acyclovir (ACV).

13. The method of claim 1, wherein said cell is a cancer cell.

14. The method of claim 13, wherein said cancer cell is a cell comprising an intraepithelial neoplasias (IN).

15. The method of claim 13, wherein said cancer cell is a cell comprising an anogenital cancer.

16. The method of claim 1, wherein said cell is a metastatic cell.

17. The method of claim 1, wherein said cell is cell in a solid tumor.

18. A nucleic acid construct comprising an HPV promoter operably linked to a heterologous effector gene.

19. The construct of claim 18, wherein said promoter is a promoter up-regulated by the HPV E2 protein.

20. The construct of claim 18, wherein said promoter is a promoter from HPV-16.

21. The construct of claim 18, wherein said promoter is a full-length HPV LCR.

22. The construct of claim 18, wherein said promoter is sufficient length of an HPV LCR to induce transcription of a nucleic acid in response to an E2 protein.

23. The construct of claim 18, wherein said promoter is an HPV16 LCR promoter.

24. The construct of claim 18, wherein said effector is selected from the group consisting of a reporter gene, a cytotoxic gene, a tumor suppressor gene, and an apoptosis gene.

25. The construct of claim 18, wherein said effector is a cytotoxic gene.

26. The construct of claim 18, wherein said effector is a cytotoxic gene selected from the group consisting of a ricin gene, an abrin gene, a pseudomonas exotoxin gene, a diphtheria toxin gene, and a thymidine kinase gene.

27. A mammalian cell comprising the construct of any one of claims claims 18 through 26.

28. A composition comprising the construct of any one of claims claims 18 through 26 in a vector selected from the group consisting of a retroviral vector, an adeno-associated vector (AAV), an adenoviral vectors, a herpes viral vector, and a Sindbis viral vector.

29. The composition of claim 28, wherein said composition is a pharmaceutical composition in unit dosage form.

30. A composition comprising the construct of any one of claims 18 through 26 in a delivery agent selected from the group consisting of a lipid, a liposome, a cationic lipid, and a dendrimer.

31. The composition of claim 30, wherein said composition is a pharmaceutical composition in unit dosage form.

32. A method of treating cells infected with HPV, said method comprising transfecting said cells with the construct of claim 25, in sufficient concentration to produce a lethal concentration of said cytotoxin in the cells.

33. The method of claim 32, further comprising contacting said cells with ganciclovir (GCV) or acyclovir (ACV).

34. A kit for selectively killing cells infected with HPV, said kit comprising a container containing the composition of any one of claims 28 through 31.

35. A method of selectively labeling a cell bearing a human papillomavirus (HPV), said method comprising:
transfecting a mammalian cell with a nucleic acid construct encoding an HPV specific promoter that is induced by an HPV protein wherein said promoter is operably linked to a nucleic acid comprising a reporter gene whereby said cell, when infected with a human papilloma virus induces expression of said reporter gene thereby labeling said mammalian cell.

36. The method of claim 35, wherein said transfecting comprises delivery using a vector selected from the group consisting of a retroviral vector, an adeno-associated vector (AAV), an adenoviral vectors, a herpes viral vector, and a Sindbis viral vector.

37. The method of claim 35, wherein said transfecting comprises use of a delivery agent selected from the group consisting of a lipid, a liposome, a cationic lipid, and a dendrimer.

38. The method of claim 35, wherein said promoter is a promoter up-regulated by the HPV E2 protein.

39. The method of claim 35, wherein said promoter is a promoter from HPV-16.

40. The method of claim 35, wherein said promoter is a full-length HPV LCR.

41. The method of claim 35, wherein said promoter is sufficient length of an HPV LCR to induce transcription of a nucleic acid in response to an E2 protein.

42. The method of claims 40 or 41, wherein said LCR is an HPV-16 LCR.

43. The method of claim 35, wherein said reporter gene is selected from the group consisting of an enzymatic reporter, a colorimetric reporter, a luminescent reporter, and a fluorescent reporter.

44. The method of claim 43, wherein said reporter gene is an Fflux gene or a green fluorescent protein gene.

45. The method of claim 35, wherein said cell is a cancer cell.

46. The method of claim 45, wherein said cancer cell is a cell comprising an intraepithelial neoplasias (EN).

47. The method of claim 45, wherein said cancer cell is a cell comprising an anogenital cancer.

48. The method of claim 35, wherein said cell is a metastatic cell.

49. The method of claim 35, wherein said cell is cell in a solid tumor.


SEQUENCE LISTING (9 sequences)

SEQ ID NO: 1 - DNA, 12 bp, Artificial
  synthetic palindromic sequence
accnnnnnnggt

SEQ ID NO: 2 - DNA, 27 bp, Artificial
  primer
cggctcgagtgtagcgccaggcccatt

SEQ ID NO: 3 - DNA, 27 bp, Artificial
  primer
cggaagcttgggtcctgaaacattgca

SEQ ID NO: 4 - DNA, 27 bp, Artificial
  primer
cggaagcttcccaggtccacttcgcat

SEQ ID NO: 5 - DNA, 27 bp, Artificial
  primer
cggtctagacatagcgcgggttccttc

SEQ ID NO: 6 - DNA, 20 bp, Artificial
  primer
cgttctggctcctcatgtcg

SEQ ID NO: 7 - DNA, 20 bp, Artificial
  primer
gccagcatagccaggtcaag

SEQ ID NO: 8 - DNA, 15 bp, Artificial
  primer
gtatgggaagttcat

SEQ ID NO: 9 - DNA, 16 bp, Artificial
  primer
cttagtggtgtggcag